(19) United States
(12) Patent Application Publication
(10) Pub. No.: US 2026/0014216 A1
(43) Pub. Date: Jan. 15, 2026

(54) USE OF PROBIOTIC COMPOSITION IN PREPARATION OF FORMULATION FOR IMPROVING VAGINAL HEALTH

(71) Applicant: SICHUAN ANAEROBIC BIOTECHNOLOGY CO., LTD., Chengdu (CN)

(72) Inventors: Lei Cheng, Chengdu (CN); Qiong Wang, Chengdu (CN); Wanqiu Zeng, Chengdu (CN)

(21) Appl. No.: 19/281,460

(22) Filed: Jul. 25, 2025

Related U.S. Application Data
(63) Continuation-in-part of application No. 17/435,670, filed on Sep. 1, 2021, now Pat. No. 12,397,024.

(30) Foreign Application Priority Data
Aug. 9, 2019 (CN) 201910732792.4
May 29, 2025 (CN) 202510706087.2
Jul. 18, 2025 (CN) 202510993059.3

(51) Int. Cl. A61K 35/747; A61K 9/16; A61K 9/19; A61K 31/4164; A61K 35/00; A61P 15/02; A61P 31/04; C12N 1/20; C12R 1/225

(52) U.S. Cl. CPC A61K 35/747; A61K 9/1623; A61K 9/19; A61K 31/4164; A61P 15/02; A61P 31/04; C12N 1/205; A61K 2035/115; C12R 2001/225

(57) ABSTRACT

The present disclosure provides use of a probiotic composition comprising active ingredients including Lactobacillus johnsonii, Lactobacillus gasseri, Lactobacillus crispatus, and Lactobacillus jensenii in the preparation of a microbial formulation, wherein the microbial formulation is used for: improving the therapeutic effect of an additional drug for treating bacterial vaginosis in the treatment of bacterial vaginosis; improving the prognosis of bacterial vaginosis treated with an additional drug for treating bacterial vaginosis; treating, preventing, mitigating, and/or controlling the recurrence of bacterial vaginosis in a subject; treating, preventing, mitigating, and/or controlling atrophic vaginitis in a subject; improving the Nugent score of a subject; or ameliorating vaginal dryness in a subject.

### CROSS-REFERENCE TO RELATED APPLICATIONS

The present application is a continuation-in-part application of U.S. patent application Ser. No. 17/435,670 filed on Sep. 1, 2021, which is a national stage application of PCT application No. PCT/CN2020/107915 filed on Aug. 7, 2020, which in turn claims the benefit of Chinese patent application No. 201910732792.4 filed on Aug. 9, 2019. The present application also claims priorities from Chinese patent application No. 202510706087.2 filed on May 29, 2025 and Chinese patent application No. 202510993059.3 filed on Jul. 18, 2025. The contents of all of the aforementioned applications are incorporated by reference herein in their entirety.

### TECHNICAL FIELD

The present disclosure pertains to the field of biopharmaceuticals, and relates to use of a probiotic composition in the preparation of a formulation for improving vaginal health.

### BACKGROUND

Globally, approximately 40% of women experience vaginitis at least once in their lifetimes. This type of disease is often accompanied by symptoms such as pruritus vulvae, abnormal secretions, dyspareunia, etc., which seriously affect the quality of life. The recurrent nature of vaginitis is more likely to lead to psychological problems such as anxiety, depression, etc., and may even cause complications such as infertility, prelabor rupture of membranes, etc. Epidemiological surveys indicate that among married women in China, the prevalence rate of gynecological diseases reaches more than 65%, showing a significant trend toward affecting younger age groups, suggesting an urgent need for prevention and treatment.

Bacterial vaginosis (BV) accounts for 30%-50% of vaginitis cases and is caused by an imbalance in the vaginal flora. Antibiotics are the first choice for clinical treatment of BV, with metronidazole and clindamycin being the primary drugs. However, this treatment regimen faces two major issues. The first is antibiotic resistance, with the resistance rate to metronidazole being up to 63.8%-67% and the resistance rate to clindamycin being about 24.1%, resulting in a reduced cure rate. The second is that although antibiotics can quickly relieve symptoms, the recurrence rate within 3 months is as high as 30%-50%. The mechanism of recurrence involves persistent depletion of Lactobacillus vaginalis, biofilm formation, and host immune abnormalities. Clinical observations have found that individuals with recurrent infections often harbor multidrug-resistant bacterial colonization, and conventional therapies struggle to restore the flora homeostasis and may even lead to a vicious cycle.

Lactobacillus, as the dominant vaginal flora, inhibits pathogen adhesion by producing lactic acid to maintain an acidic environment (pH 3.8-4.5), generating H₂O₂ and bacteriocins, and the like. Studies have shown that supplementation with exogenous lactobacilli (e.g., Lactobacillus crispatus and Lactobacillus gasseri) can significantly improve the vagina's ability to regulate pH and maintain the vaginal microenvironment. Live bacterial drugs for BV treatment on the market in China include Lacidophilin Vaginal Capsules (Yanhua) produced by Xi'an Zhenghao Biological Pharmaceutical Co., Ltd. and Live Lactobacillus Capsule for Vaginal Use (Dingjunsheng) produced by Inner Mongolia Shuangqi Pharmaceutical Co., Ltd. Live bacterial drugs marketed outside of China include LJ LACTO (Lactobacillus plantarum P 17630), but there are no reports on its therapeutic effect in treating BV and recurrent cases. Live bacterial drugs currently in the clinical stage include Lactobacillus crispatus Lactin-V.

The clinical research findings for these live bacterial drugs include the following: In the study by Wu Xiaohui et al. (Effect of Lacidophilin Vaginal Capsules in Combination with Nifuratel on Symptom Amelioration and Microinflammatory Response Indicators in Patients with Bacterial Vaginosis, Heilongjiang Medicine Journal, 2024 (6)), BV patients treated with Lacidophilin Vaginal Capsules in combination with the antibiotic nifuratel showed a cure rate of 18.52% and a 3-month recurrence rate of 20%. In the study by He Liangliang et al. (Effect of Lacidophilin Vaginal Capsules in Combination with Nifuratel Tablets on Vaginal Microenvironment and Recurrence in Elderly Patients with Bacterial Vaginosis, Reflexology and Rehabilitation Medicine, 2024,5(23)), elderly BV patients treated with Lacidophilin Vaginal Capsules in combination with nifuratel showed a recovery rate of 24.44% and a 2-month recurrence rate of 9.1%. In the study by Liao Xixi et al. (Clinical Efficacy Observation of Live Lactobacillus Capsule in Combination with Topical Metronidazole in Treatment of Bacterial Vaginosis, Modern Diagnosis & Treatment, 2024 Nov 35(22)), no cure indicators were observed, and the analysis focused solely on data such as marked efficacy (the markedly effective rate was 57%, which was approximately 12% higher than that in the metronidazole monotherapy group). Similarly, in the study by Niu Huihui (Efficacy of Live Lactobacillus Capsule in Combination with Metronidazole in Treating Patients with Bacterial Vaginosis, Medical Journal of Chinese People's Health, 2025 Feb 37(3)), no cure indicators were observed, and regarding the markedly effective rate, the combination therapy group showed approximately 10% improvement compared to the antibiotic monotherapy group. Craig R Cohen et al. (Randomized Trial of Lactin-V to Prevent Recurrence of Bacterial Vaginosis. N Engl J Med. 2020 May 14; 382(20):1906-1915) observed the therapeutic effect of Lactin-V in combination with metronidazole versus placebo in combination with metronidazole in BV treatment in a randomized, double-blind, placebo-controlled phase II clinical study; in the study, the cure rate data were not reported; at week 12, the recurrence rate was 30% in the Lactin-V group and 45% in the placebo group, indicating that Lactin-V reduced the recurrence rate by 15%; at week 24, the recurrence rate was 39% in the Lactin-V group and 54% in the placebo group.

It can be seen that microecological preparations can improve the therapeutic outcomes of BV. However, more effective drugs are still required for both cure rate improvement and recurrence mitigation of BV. The further benefits of live bacteria for vaginitis in women remain to be explored.

### SUMMARY

In order to solve the problems in the prior art, the present disclosure provides a method for improving vaginal health status, comprising administering to a subject a therapeutically effective amount of a microbial formulation, wherein the microbial formulation comprises a probiotic composition comprising active ingredients comprising Lactobacillus johnsonii, Lactobacillus gasseri, Lactobacillus crispatus, and Lactobacillus jensenii, and the improving vaginal health status is selected from:
a): improving the therapeutic effect of an additional drug for treating bacterial vaginosis in the treatment of bacterial vaginosis;
b): improving the prognosis of bacterial vaginosis treated with an additional drug for treating bacterial vaginosis;
c): treating, preventing, mitigating, and/or controlling the recurrence of bacterial vaginosis in the subject;
d): treating, preventing, mitigating, and/or controlling postmenopausal atrophic vaginitis in the subject;
e): improving the Nugent score of the subject;
f): ameliorating vaginal dryness in the subject; and
g): regulating the vaginal microbiota of the subject.

In some embodiments, the microbial formulation is used in combination with an additional drug for treating bacterial vaginosis, the additional drug for treating bacterial vaginosis including an antibiotic, boric acid, and/or a traditional Chinese medicine formulation.

In some embodiments, the microbial formulation is used in combination with an antibiotic for treating bacterial vaginosis.

In some embodiments, the antibiotic is selected from one or more of metronidazole, tinidazole, ornidazole, clindamycin, and fluconazole.

In some embodiments, the additional drug for treating bacterial vaginosis is administered to the subject prior to the administration of the microbial formulation to the subject.

In some embodiments, an antibiotic treatment is first administered to the subject, and after the antibiotic treatment is completed, the microbial formulation is administered to the subject for 10-18 consecutive days, with each of the strains in the microbial formulation administered in an amount of 10⁶ CFU to 10¹³ CFU per day.

In some embodiments, an antibiotic treatment is first administered to the subject, and after the antibiotic treatment is completed, the microbial formulation is administered to the subject once daily for 10-18 consecutive days, preferably 14 consecutive days, with each of the strains administered in an amount of 10⁶ CFU to 10¹³ CFU per administration.

In some embodiments, the Lactobacillus johnsonii is: Lactobacillus johnsonii with a microbial deposit number of CCTCC M 2019426, a pure culture thereof, a passaged strain thereof, or a clonal strain thereof; Lactobacillus johnsonii having 96% or more, 97% or more, 98% or more, or 99% or more ANI and substantially identical genetic characteristics to the strain with the microbial deposit number of CCTCC M 2019426; or Lactobacillus johnsonii having a substantially identical genomic sequence to the strain with the microbial deposit number of CCTCC M 2019426;

the Lactobacillus gasseri is: Lactobacillus gasseri with a microbial deposit number of CCTCC M 2019430, a pure culture thereof, a passaged strain thereof, or a clonal strain thereof; Lactobacillus gasseri having 96% or more, 97% or more, 98% or more, or 99% or more ANI and substantially identical genetic characteristics to the strain with the microbial deposit number of CCTCC M 2019430; or Lactobacillus gasseri having a substantially identical genomic sequence to the strain with the microbial deposit number of CCTCC M 2019430;
the Lactobacillus crispatus is: Lactobacillus crispatus with a microbial deposit number of CCTCC M 2019427, a pure culture thereof, a passaged strain thereof, or a clonal strain thereof; Lactobacillus crispatus having 96% or more, 97% or more, 98% or more, or 99% or more ANI and substantially identical genetic characteristics to the strain with the microbial deposit number of CCTCC M 2019427; or Lactobacillus crispatus having a substantially identical genomic sequence to the strain with the microbial deposit number of CCTCC M 2019427;
the Lactobacillus jensenii is: Lactobacillus jensenii with a microbial deposit number of CCTCC M 2019429, a pure culture thereof, a passaged strain thereof, or a clonal strain thereof; Lactobacillus jensenii having 96% or more, 97% or more, 98% or more, 99% or more ANI and substantially identical genetic characteristics to the strain with the microbial deposit number of CCTCC M 2019429; or Lactobacillus jensenii having a substantially identical genomic sequence to the strain with the microbial deposit number of CCTCC M 2019429.

The passaged strains or passaged cells described herein refer to those derived from the passaging process with no changes in the promoter activity of all genes, no changes in the transcription initiation and transcription termination of all genes, no changes in the amino acid sequences of all proteins, and no changes in the bacterial reproductive capability.

In some embodiments, the subject is a human female.

In some embodiments, based on CFU, in the probiotic composition, any two of the bacteria are in an amount ratio of (1-100):(1-100), preferably (1-10):(1-10), and more preferably 1:(1-10).

In some embodiments, in a unit formulation of the microbial formulation, the total viable count of the bacteria is not less than 1×10⁷ CFU, and the viable count of each of the strains is not less than 1×10⁶ CFU.

In some embodiments, the microbial formulation further comprises a pharmaceutically acceptable excipient.

In some embodiments, the probiotic composition is a lyophilized powder.

In some embodiments, the excipient includes a lyoprotectant and a diluent.

In some embodiments, the microbial formulation is in a dosage form selected from: a capsule, a tablet, and a lyophilized powder.

In some embodiments, in a unit formulation of the microbial formulation, the viable count of each of the strains is 1×10⁶ CFU to 1×10¹³ CFU.

In some embodiments, in a unit formulation of the microbial formulation, the viable count of each of the strains is (1-10)×10⁶ CFU, or (1-10)×10⁷ CFU, or (1-10)×10⁸ CFU, or (1-10)×10⁹ CFU, or (1-10)×10¹⁰ CFU, or (1-10)×10¹¹ CFU, or (1-10)×10¹² CFU.

In some embodiments, regulating the vaginal microbiota of the subject comprises increasing the relative abundance of vaginal Lactobacillus, increasing the relative abundance of vaginal Lactobacillus other than Lactobacillus iners, and/or decreasing the relative abundance of vaginal Lactobacillus iners in the subject.

In another aspect, the present disclosure provides a probiotic composition, comprising active ingredients including Lactobacillus johnsonii, Lactobacillus gasseri, Lactobacillus crispatus, and Lactobacillus jensenii, wherein the probiotic composition is a lyophilized powder.

The probiotic composition is also known as the multi-lactobacillus composition in the present disclosure.

In some embodiments, the Lactobacillus johnsonii is: Lactobacillus johnsonii with a microbial deposit number of CCTCC M 2019426, a pure culture thereof, a passaged strain thereof, or a clonal strain thereof; or Lactobacillus johnsonii having 96% or more, 97% or more, 98% or more, or 99% or more ANI and substantially identical genetic characteristics to the strain with the microbial deposit number of CCTCC M 2019426; or Lactobacillus johnsonii having a substantially identical genomic sequence to the strain with the microbial deposit number of CCTCC M 2019426;

the Lactobacillus gasseri is: Lactobacillus gasseri with a microbial deposit number of CCTCC M 2019430, a pure culture thereof, a passaged strain thereof, or a clonal strain thereof; or Lactobacillus gasseri having 96% or more, 97% or more, 98% or more, or 99% or more ANI and substantially identical genetic characteristics to the strain with the microbial deposit number of CCTCC M 2019430; or Lactobacillus gasseri having a substantially identical genomic sequence to the strain with the microbial deposit number of CCTCC M 2019430;
the Lactobacillus crispatus is: Lactobacillus crispatus with a microbial deposit number of CCTCC M 2019427, a pure culture thereof, a passaged strain thereof, or a clonal strain thereof; or Lactobacillus crispatus having 96% or more, 97% or more, 98% or more, or 99% or more ANI and substantially identical genetic characteristics to the strain with the microbial deposit number of CCTCC M 2019427; or Lactobacillus crispatus having a substantially identical genomic sequence to the strain with the microbial deposit number of CCTCC M 2019427;
the Lactobacillus jensenii is: Lactobacillus jensenii with a microbial deposit number of CCTCC M 2019429, a pure culture thereof, a passaged strain thereof, or a clonal strain thereof; or Lactobacillus jensenii having 96% or more, 97% or more, 98% or more, or 99% or more ANI and substantially identical genetic characteristics to the strain with the microbial deposit number of CCTCC M 2019429; or Lactobacillus jensenii having a substantially identical genomic sequence to the strain with the microbial deposit number of CCTCC M 2019429.

In yet another aspect, the present disclosure provides a microbial formulation, comprising the Lactobacillus johnsonii, the Lactobacillus gasseri, the Lactobacillus crispatus, and the Lactobacillus jensenii described above, with each strain having a viable count of 1×10⁶ CFU to 1×10¹³ CFU per unit formulation.

The foregoing various lactobacilli with specific microbial deposit numbers are screened and separated from the vaginas of the health females in China. Under the provisions of the Budapest Treaty on the International Recognition of the Deposit of Microorganisms for the Purposes of Patent Procedure, the foregoing various lactobacilli were deposited with the international depositary authority: China Center for Type Culture Collection (CCTCC), Wuhan University, Wuhan 430072 P.R. China, on Jun. 4, 2019. The Lactobacillus johnsonii with the microbial deposit number of CCTCC M 2019426 is also referred to as strain Ljohn-1 in the present disclosure; the Lactobacillus gasseri with the microbial deposit number of CCTCC M 2019430 is also referred to as strain Lgass-17 in the present disclosure; the Lactobacillus crispatus with the microbial deposit number of CCTCC M 2019427 is also referred to as strain Lcris-2 in the present disclosure; and the Lactobacillus jensenii with the microbial deposit number of CCTCC M 2019429 is also referred to as strain Ljen-10 in the present disclosure. The Lactobacillus rhamnosus with the microbial deposit number of CCTCC M 2019428 is also referred to as strain Lrham-6 in the present disclosure. All restrictions imposed by the depositor on the availability to the public of the deposited material will be irrevocably removed upon the granting of a patent.

After being uploaded to EzBiocloud, a complete genome sequence of the Lactobacillus rhamnosus Lrham-6 is compared with all complete genome sequences of conspecies that it can link to obtain an average nucleotide identity (ANI) ratio as shown in Table 1, proving that it is a new Lactobacillus strain.

TABLE 1
ANI Comparison of Complete Genomes of Lrham-6 and Different Lactobacillus Rhamnosus

| Name of | Species of | | |
|---|---|---|---|
| sequential document | reference document | Name of reference document | ANI (%) |
| Lrham- | Lactobacillus | GCA_002406795.1_ASM240679v1_genomic.fna.gz | 99.9524 |
| 6.genomic.fasta | rhamnosus | | |
| Lrham- | Lactobacillus | GCA_002406745.1_ASM240674v1_genomic.fna.gz | 99.9515 |
| 6.genomic.fasta | rhamnosus | | |
| Lrham- | Lactobacillus | GCA_002406715.1_ASM240671v1_genomic.fna.gz | 99.9506 |
| 6.genomic.fasta | rhamnosus | | |
| Lrham- | Lactobacillus | GCA_000173255.2_ASM17325v2_genomic.fna.gz | 99.9388 |
| 6.genomic.fasta | rhamnosus | | |
| Lrham- | Lactobacillus | GCA_002406785.1_ASM240678v1_genomic.fna.gz | 99.9366 |
| 6.genomic.fasta | rhamnosus | | |
| Lrham- | Lactobacillus | GCA_001812155.1_ASM181215v1_genomic.fna.gz | 99.8953 |
| 6.genomic.fasta | rhamnosus | | |
| Lrham- | Lactobacillus | GCA_001062955.1_ASM106295v1_genomic.fna.gz | 99.7943 |
| 6.genomic.fasta | rhamnosus | | |
| Lrham- | Lactobacillus | GCA_001064785.1_ASM106478v1_genomic.fna.gz | 99.7669 |
| 6.genomic.fasta | rhamnosus | | |
| Lrham- | Lactobacillus | GCA_001657135.1_ASM165713v1_genomic.fna.gz | 99.6405 |
| 6.genomic.fasta | rhamnosus | | |
| Lrham- | Lactobacillus | GCA_001062885.1_ASM106288v1_genomic.fna.gz | 99.6173 |
| 6.genomic.fasta | rhamnosus | | |

After being uploaded to EzBiocloud, a complete genome sequence of the Lactobacillus gasseri Lgass-17 is compared with all complete genome sequences of; conspecies that it can link to obtain an average nucleotide identity (ANI) ratio as shown in Table 2, proving that it is a new Lactobacillus strain.

TABLE 2
ANI Comparison of Complete Genomes of Lgass-17 and Different Lactobacillus Gasseri

| Name of | Species of | | |
|---|---|---|---|
| sequential document | reference document | Name of reference document | ANI (%) |
| Lgass- | Lactobacillus | GCA_000177035.2_ASM17703v2_genomic.fna.gz | 99.9256 |
| 17.genomic.fasta | gasseri | | |
| Lgass- | Lactobacillus | GCA_000177415.1_ASM17741v1_genomic.fna.gz | 99.8912 |
| 17.genomic.fasta | gasseri | | |
| Lgass- | Lactobacillus | GCA_000176995.2_ASM17699v2_genomic.fna.gz | 99.7935 |
| 17.genomic.fasta | gasseri | | |
| Lgass- | Lactobacillus | GCA_001063045.1_ASM106304v1_genomic.fna.gz | 99.776 |
| 17.genomic.fasta | gasseri | | |
| Lgass- | Lactobacillus | GCA_001066235.1_ASM106623v1_genomic.fna.gz | 99.7531 |
| 17.genomic.fasta | gasseri | | |
| Lgass- | Lactobacillus | GCA_000439915.1_L.gasseri_2016_V1_genomic.fna.gz | 99.7284 |
| 17.genomic.fasta | gasseri | | |
| Lgass- | Lactobacillus | GCA_001063065.1_ASM106306v1_genomic.fna.gz | 99.7232 |
| 17.genomic.fasta | gasseri | | |
| Lgass- | Lactobacillus | GCA 000014425.1_ASM1442v1_genomic.fna.gz | 99.6302 |
| 17.genomic.fasta | gasseri | | |
| Lgass- | Lactobacillus | GCA_000155935.2_Lacto_gasseri_MV-22_V2_genomic.fna.gz | 99.5271 |
| 17.genomic.fasta | gasseri | | |
| Lgass- | Lactobacillus | GCA_000283135.1_ASM28313v1_genomicfna.gz | 99.2187 |
| 17.genomic.fasta | gasseri | | |

After being uploaded to EzBiocloud, a complete genome sequence of the Lactobacillus johnsonii Ljohn-1 is compared with all complete genome sequences of conspecies that it can link to obtain an average nucleotide identity (ANI) ratio as shown in Table 3, proving that it is a new Lactobacillus strain.

TABLE 3
ANI Comparison of Complete Genomes of Ljohn-1 and Different Lactobacillus Johnsonii

| Name of | Species of | | |
|---|---|---|---|
| sequential document | reference document | Name of reference document | ANI (%) |
| Ljohn- | Lactobacillus | GCA_000498675.1_ASM49867v1_enomic.fna.gz | 95.5668 |
| 1.genomic.fasta | johnsonii | | |
| Ljohn- | Lactobacillus | GCA_002253275.1_ASM225327v1_genomic.fna.gz | 95.4233 |
| 1.genomic.fasta | johnsonii | | |
| Ljohn- | Lactobacillus | GCA_002253205.1_ASM225320v1_genomic.fna.gz | 95.3823 |
| 1.genomic.fasta | johnsonii | | |
| Ljohn- | Lactobacillus | GCA_002253165.1_ASM225316v1_genomic.fna.gz | 95.3732 |
| 1.genomic.fasta | johnsonii | | |
| Ljohn- | Lactobacillus | GCA_002253185.1_ASM225318v1_genomic.fna.gz | 95.3683 |
| 1.genomic.fasta | johnsonii | | |
| Ljohn- | Lactobacillus | GCA_002253245.1_ASM225324v1_genomic.fna.gz | 95.3515 |
| 1.genomic.fasta | johnsonii | | |
| Ljohn- | Lactobacillus | GCA_002253285.1_ASM225328v1_genomic.fna.gz | 95.3379 |
| 1.genomic.fasta | johnsonii | | |
| Ljohn- | Lactobacillus | GCA_001270785.1_ASM127078v1_genomic.fna.gz | 95.3079 |
| 1.genomic.fasta | johnsonii | | |
| Ljohn- | Lactobacillus | GCA_001572665.1_ASM157266v1_genomic.fna.gz | 95.1633 |
| 1.genomic.fasta | johnsonii | | |
| Ljohn- | Lactobacillus | GCA_002803395.1_ASM280339v1_genomic.fna.gz | 95.0963 |
| 1.genomic.fasta | johnsonii | | |

After being uploaded to EzBiocloud, a complete genome sequence of the Lactobacillus jensenii Ljen-10 is compared with all complete genome sequences of conspecies that it can link to obtain an average nucleotide identity (ANI) ratio as shown in Table 4, proving that it is a new Lactobacillus strain.

TABLE 4
ANI Comparison of Complete Genomes of Ljen-10 and Different Lactobacillus Jensenii

| Name of | Species of | | |
|---|---|---|---|
| sequential document | reference document | Name of reference document | ANI (%) |
| Ljen- | Lactobacillus | GCA_002848045.1_ASM284804v1_genomic.fna.gz | 88.9784 |
| 10.genomic.fasta | jensenii | | |
| Ljen- | Lactobacillus | GCA_000155915.2_Lacto_jensenii_1153_v2_genomic.fna.gz | 88.7036 |
| 10.genomic.fasta | jensenii | | |
| Ljen- | Lactobacillus | GCA_001936235.1_ASM193623v1_genomic.fna.gz | 88.6759 |
| 10.genomic.fasta | jensenii | | |
| Ljen- | Lactobacillus | GCA_002863405.1_ASM286340v1_genomic.fna.gz | 88.6432 |
| 10.genomic.fasta | jensenii | | |
| Ljen- | Lactobacillus | GCA_000466805.1_ASM46680v1_genomic.fna.gz | 88.5848 |
| 10.genomic.fasta | jensenii | | |
| Ljen- | Lactobacillus | GCA_000162335.1_ASM16233v1_genomic.fna.gz | 88.5791 |
| 10.genomic.fasta | jensenii | | |
| Ljen- | Lactobacillus | GCA_000175035.1_ASM17503v1_genomic.fna.gz | 88.5055 |
| 10.genomic.fasta | jensenii | | |
| Ljen- | Lactobacillus | GCA_001436455.1_ASM143645v1_genomic.fna.gz | 88.4346 |
| 10.genomic.fasta | jensenii | | |
| Ljen- | Lactobacillus | GCA_001012665.1_IM18-1v1_genomic.fna.gz | 88.4042 |
| 10.genomic.fasta | jensenii | | |
| Ljen- | Lactobacillus | GCA_001012685.1_IM18-3v1_genomic.fna.gz | 88.3924 |
| 10.genomic.fasta | jensenii | | |

After being uploaded to EzBiocloud, a complete genome sequence of the Lactobacillus crispatus Lcris-2 compared with all complete genome sequences of conspecies that it can link to obtain an average nucleotide identity (ANI) ratio as shown in Table 5, proving that it is a new Lactobacillus strain.

TABLE 5
ANI Comparison of Complete Genomes of Lcris-2 and Different Lactobacillus Crispatus

| Name of | Species of | | |
|---|---|---|---|
| sequential document | reference document | Name of reference document | ANI (%) |
| Lcris- | Lactobacillus | GCA_002861765.1_ASM286176v1_genomic.fna.gz | 99.3638 |
| 2.genomic.fasta | crispatus | | |
| Lcris- | Lactobacillus | GCA_001546015.1_ASM154601v1_genomic.fna.gz | 99.2264 |
| 2.genomic.fasta | crispatus | | |
| Lcris- | Lactobacillus | GCA_000176975.2_ASM17697v2_genomic.fna.gz | 99.2008 |
| 2.genomic.fasta | crispatus | | |
| Lcris- | Lactobacillus | GCA_001546025.1_ASM154602v1_genomic.fna.gz | 99.1693 |
| 2.genomic.fasta | crispatus | | |
| Lcris- | Lactobacillus | GCA_001541585.1_ASM154158v1_genomic.fna.gz | 99.0587 |
| 2.genomic.fasta | crispatus | | |
| Lcris- | Lactobacillus | GCA_001541515.1_ASM154151v1_genomic.fna.gz | 99.0447 |
| 2.genomic.fasta | crispatus | | |
| Lcris- | Lactobacillus | GCA_000301135.1_Lact_cris_FB077-07_V1_genomic.fna.gz | 99.0328 |
| 2.genomic.fasta | crispatus | | |
| Lcris- | Lactobacillus | GCA_002861775.1_ASM286177v1_genomic.fna.gz | 99.0016 |
| 2.genomic.fasta | crispatus | | |
| Lcris- | Lactobacillus | GCA_000466885.2_ASM46688v2_genomic.fna.gz | 98.9705 |
| 2.genomic.fasta | crispatus | | |
| Lcris- | Lactobacillus | GCA_000301115.1_Lact_cris_FB049-03_V1_genomic.fna.gz | 98.9698 |
| 2.genomic.fasta | crispatus | | |

A viable count of the multi-lactobacillus composition is 10⁵-10¹¹ CFU/g, and a content of each single bacteria is not lower than 10⁵ CFU/g.

According to the present disclosure, a bacteriological preparation with the above multi-lactobacillus composition as an active ingredient may be a suspension or lyophilized bacteria powder.

According to the present disclosure, the multi-lactobacillus composition is applied to preparation of drugs or health care products for preventing or treating pathogenic bacteria of bacterial vaginosis (BV).

According to the present disclosure, the multi-lactobacillus composition may be applied to preparation of sanitary articles for pudendum, such as sanitary napkins, tampons or health care solution for pudendum.

According to the present disclosure, the multi-lactobacillus composition may be applied to preparation of drugs or health care products for adjusting the balance of vaginal flora.

According to the present disclosure, the multi-lactobacillus composition may be applied to preparation of drugs or health care products with an adhesion function of vaginal epithelial cells.

According to the present disclosure, the multi-lactobacillus composition may be applied to preparation of drugs, health care products and food additives for preventing and treating the pathogenic bacteria, and the pathogenic bacteria include but not limited to any one or more of Gardnerella vaginalis, Staphylococcus aureus, Pseudomonas aeruginosa, Escherichia coli, Salmonella paratyphi B and Shigella dysenteriae.

The multi-lactobacillus composition may be also applied to preparation of external care products for infants delivered by caesarean section. The infants delivered by caesarean section will not get exogenous probiotics during their birth because they are not born through female vagina. For this reason, the probiotics screened from the vaginas of the females may be prepared into external care products to daub or wash the infant bodies.

Compared with the prior art, the present disclosure has the following beneficial effects:

With diverse strains of lactobacilli colonized in the vaginas of the health females, different strains of different or same species have different probiotic abilities. Hence, there is a necessity to take into comprehensive consideration the varieties of lactobacilli, lactic acid and hydrogen peroxide production capacities, inhibition of pathogenic bacteria and adhesion on vaginal cells while considering the vaginal lactobacillus probiotics. In order to cover as many females with vaginal dysbacteriosis as possible, and meanwhile, strengthen the ability to inhibit different pathogenic bacteria, the inventor will carry out reasonable compatibility for proper strains from a variety of strains separated and screened from the health females in China, and use the separated and screened lactobacilli to prevent and treat the BV by virtue of three or more Lactobacillus combination formulas. Through experimental verification, the multi-lactobacillus composition has the better probiotic ability, and belongs to the dominant strain in the vaginas of the females in China. Accordingly, it is expected that the multi-lactobacillus composition will have the better effect to treat and prevent vaginal diseases of Chinese females, and also has a wide cover range.

The microbial formulation of the present disclosure exhibits good safety and has a surprising therapeutic effect. It can significantly improve the cure rate of patients with bacterial vaginosis (BV). When used sequentially with an antibiotic, it can improve the clinical cure rate to no less than 90%, and can also reduce the recurrence rate. The 3-month recurrence rate is less than 10%, representing a reduction of no less than 30% compared to antibiotic monotherapy.

### BRIEF DESCRIPTION OF DRAWINGS

FIGS. 1A and 1B are diagrams that show comparison of rating of hydrogen peroxide production capacities of single bacteria and combined bacteria.

FIGS. 2A and 2B are diagrams that show comparison of lactic acid production capacities of single bacteria and combined bacteria.

FIGS. 3A and 3B are diagrams that show comparison of inhibitions on Gardnerella vaginalis by single bacteria and combined bacteria.

FIGS. 4A and 4B are diagrams that show comparison of adhesions on hela cells by single bacteria and combined bacteria.

FIGS. 5A and 5B are diagrams that show comparison of inhibitions on Staphylococcus aureus by single bacteria and combined bacteria.

FIGS. 6A and 6B are diagrams that show comparison of inhibitions on Escherichia coli by single bacteria and combined bacteria.

FIGS. 7A and 7B are diagrams that show comparison of inhibitions on Pseudomonas aeruginosa by single bacteria and combined bacteria.

FIGS. 8A and 8B are diagrams that show comparison of inhibitions on Shigella dysenteriae by single bacteria and combined bacteria.

FIGS. 9A and 9B are diagrams that show comparison of inhibitions on Salmonella paratyphi B by single bacteria and combined bacteria.

FIG. 10 is a test diagram 1 of a solid symbiotic effect between all single bacteria in multi-lactobacillus.

FIG. 11 is a test diagram 2 of a symbiotic effect of a broth between all single bacteria in multi-lactobacillus.

### DETAILED DESCRIPTION

### Terminology

“Bacterial vaginosis”, also known as “BV”, is a vaginal infection typically caused by a decrease or disappearance of the normal hydrogen peroxide-producing lactobacilli in the vagina, and an increase in facultative anaerobic and anaerobic bacteria. Common pathogens include facultative anaerobic bacteria (Gardnerella vaginalis) and anaerobic bacteria (Prevotella, Mobiluncus, Bacteroides, and Atopobium vaginae), as well as Ureaplasma urealyticum, Mycoplasma hominis, etc.

“Recurrence” refers to the reappearance of symptoms and pathological manifestations of a disease after the disease has been treated with symptomatic relief or disappearance and maintained a stable condition for a period of time, due to incomplete eradication of the original causative factors, recurrent imbalance in the body's microenvironment, or the like.

“Postmenopausal atrophic vaginitis” refers to an inflammatory reaction (dominated by aerobic bacterial infection) occurring in women during the menopausal transition or postmenopausal period, where ovarian function decline causes a significant decrease in the levels of estrogen and other sex hormones, leading to atrophy of vaginal mucosa, changes in local microenvironment, an increase in intravaginal pH value, and a decrease in local immunity.

“Therapeutically effective amount” refers to a dose or amount of a substance (e.g., a drug or an active ingredient) sufficient to prevent, treat, alleviate, or ameliorate a particular disease or symptom.

“Unit formulation” refers to an independent dosage unit of a drug or formulation prepared according to an established specification, such as a tablet, a capsule, or an injection, where each unit contains clearly specified pharmaceutical ingredients and contents to meet precise dosing and usage requirements in clinical medication.

“Average nucleotide identity” (ANI) refers to the average percentage of nucleotide matches calculated by aligning all homologous gene sequences between two genomes (typically based on the BLAST or MUMmer algorithm). Strains with high ANI values generally share metabolic pathways and virulence genes. The species demarcation threshold generally accepted by the International Committee on Systematics of Prokaryotes (ICSP) and mainstream literature is 95%-96%. For example, Escherichia coli and Shigella exhibit an ANI of about 89%, classifying them as distinct genera. Subspecies of Klebsiella pneumoniae show an ANI of >96%, but its ANI to closely related species (e.g., K. variicola) is <95%.

Strain having a “substantially identical genomic sequence” means that the overall similarity of two strains in nucleotide sequence alignment reaches a high matching level, allowing for reasonable deviations caused by routine technical tolerances in the art (e.g., sequencing accuracy limitations, algorithmic differences in alignment, parameter settings in data analysis, etc.). The specific determination should be based on the universally recognized standards in the art (e.g., ANI thresholds commonly adopted in microbial classification), while also considering the limitations of the detection method (e.g., the error rate of whole genome sequencing and algorithmic differences in alignment tools).

“Passaged strain” refers to a strain obtained by manually transferring the strain from an original culture to a new culture medium for continuous cultivation. This process is called “passaging”, with the purpose of maintaining the survival and reproduction of the microorganisms or conducting subsequent research. Passaged strains include non-mutated strains and mutated strains, with the latter further divided into passaged strains with minor mutations but no substantial changes in toxicity (or therapeutic activity), immunogenicity, and bioactivity, and passaged strains with relatively significant mutations resulting in substantial changes in toxicity, immunogenicity, or bioactivity. The “passaged strain” claimed for protection in the present disclosure encompasses passaged strains without mutations, and passaged strains that have accumulated minor mutations but have no substantial changes in toxicity, immunogenicity, and bioactivity. As is known in the art, multiple rounds of passaging applications inevitably introduce minor mutations into strains. When the mutation occurs in a non-coding sequence region, or is a synonymous mutation in a coding region, or is a mutation that does not affect the toxicity, immunogenicity, and bioactivity of the strains (e.g., it may be a linker amino acid residue between two domains, or a residue of a minor mutation located within the higher-order structure of a protein that does not affect the toxicity, immunogenicity, and bioactivity due to the absence of contact with immune cells), it can be reasonably expected that the purposes of the present disclosure can still be achieved if these minor changes do not significantly affect the toxicity (or therapeutic activity), immunogenicity, and bioactivity of the progeny strains, and these minor changes are derived from the strains contributed by the present disclosure and thus still fall within the substantial technical contribution scope of the present disclosure. These minor mutations are still insubstantial mutations, and the strains with these minor mutations should be considered as mutated strains that have no changes in toxicity (or therapeutic activity), immunogenicity, and bioactivity. The absence of substantial changes in toxicity (or therapeutic activity), immunogenicity, and bioactivity includes, but is not limited to, circumstances where the toxicity, immunogenicity, and bioactivity are considered unchanged within the scope of limitations of detection techniques (such as detection sensitivity and detection limits), and within the range of acceptable or inevitable errors. When the toxicity, immunogenicity, and bioactivity of the progeny of the strains are determined using cells, animals, etc., differences reflected in cell lines, animal species, age, sex, health status, culture conditions, etc., as well as expected or inevitable systematic errors, fall under the scope of having no substantial changes.

In order to define the passaged strains more specifically and more clearly, the present disclosure requests protection for the passaged strains with “no changes in the promoter activity of all genes, no changes in the transcription initiation and transcription termination of all genes, no changes in the amino acid sequences of all proteins, and no changes in the bacterial reproductive capability during passaging”.

During the passaging of passaged cells, the absence of changes in the bacterial reproductive capability primarily means that the genomic sequence does not undergo any mutation and thus maintains the original reproductive capability, or minor alterations in the reproductive capability fall within the detection system error range of conventional techniques or constitute changes undetectable by conventional techniques. According to the general knowledge in the art, even for two daughter bacteria derived from a single colony of a monoclonal strain, there are systematic errors in the determination results of reproductive capability indicators (for example, there are errors in the bacterial plate counting method itself). Obviously, minor mutations inevitably occur during bacterial passaging; synonymous mutations in coding sequences or minor mutations in genomic loci that are not involved in bacterial genetic regulation do not affect the reproductive capability or bioactivity of bacteria. Such passaged bacteria fall within the substantial technical contribution scope of the deposited bacterial strains, constituting an inevitable and reasonable variation range for the direct technical contribution of the present disclosure. This range will minimize deviation between the content protected by the patent and the content defined in the claims.

The absence of changes in the promoter activity of all genes during the passaging of passaged cells primarily means that all promoters show no changes in the mode of accepting endogenous gene expression regulation within bacteria, no detectable changes in the mode of responding to external signals received, and no detectable changes in signal transduction pathway and metabolic regulation mode. Minor mutations that are not involved in regulation, such as minor base changes in the linker region between two adjacent regulatory factor binding regions of a promoter, or minor mutations in cis-acting elements that do not affect the expression regulation patterns of corresponding genes, fall under the scope of having no changes in the promoter activity.

The absence of changes in the transcription initiation and transcription termination of all genes during the passaging of passaged cells primarily means that there are no changes in the expression regulation patterns of the genes, for example, the originally inactive genes remain inactive as before, and no detectably significant changes occur in the transcriptional regulation of the originally active genes.

The absence of changes in the amino acid sequences of all proteins during the passaging of passaged cells primarily means that throughout the strain's passaging process, no changes occur in the amino acid sequences of any proteins. Synonymous mutations in genes do not affect gene functionality, while mutations during bacterial passaging are inherently inevitable. Thus, the daughter bacteria having no changes in the amino acid sequences of all proteins still fall within the technical contribution scope of the deposited strains.

Strain having “substantially identical genetic characteristics” primarily means that a passaged strain exhibits a minor gene mutation compared to a parent strain, and the daughter strain is a strain having identical genetic characteristics to the parent strain. This includes, for instance, passaged strains with “no changes in the promoter activity of all genes, no changes in the transcription initiation and transcription termination of all genes, no changes in the amino acid sequences of all proteins, and no changes in the bacterial reproductive capability during passaging”, notwithstanding minimal variations existing between such passaged strains and the parent strains. In particular, the ANI between parent and daughter strains is greater than the maximum ANI between the parent or daughter strain and strains with publicly available genomic sequences.

Strain having “substantially identical genetic characteristics” also refers to a strain that is highly homologous in evolution to the compared strain, such as a strain having minor genomic sequence or locus variations relative to the compared strain while sharing a common ancestral strain; in particular, the ANI between them is greater than the maximum ANI between the compared strain and strains with publicly available genomic sequences.

“Clonal strain” primarily refers to a genetically identical progeny strain derived directly through asexual reproduction (such as cell division or gene replication) from a single parent strain or a daughter strain thereof.

“Pure culture” primarily refers to a homogeneous culture obtained through artificial isolation and purification techniques, which is formed by the reproduction of a single cell or spore of a single microorganism (e.g., bacterium, fungus, virus, etc.) and does not contain any other contaminating bacteria or microorganisms.

### Example 1: Preparation of Microbial Formulations (Probiotic Composition Live Bacteria Capsules)

### (I) Sources of Strains

A method for screening and separating Lactobacillus johnsonii Ljohn-1, Lactobacillus gasseri Lgass-17, Lactobacillus crispatus Lcris-2, Lactobacillus jensenii Ljen-10, and Lactobacillus rhamnosus Lrham-6 is as follows:

Collecting a vaginal secretion sample of a female, aged 20-40, passing physical examination in China with a vaginal cotton swab; filling 2 mL of sterile anaerobic PBS buffer solution into an anaerobic tube with the cotton swab, shaking fully for mixing uniformly, and diluting as a stock solution by ten consecutive gradients; coating 100 μL of liquid diluted to 10,000 times to a MRS solid medium, cultivating in an anaerobic incubator at 37° C., selecting a single colony of suspected lactobacillus to cultivate for 24 h in a MRS broth medium after cultivating for 48 h, continuously transferring one part of bacteria solution obtained upon cultivation for continuous cultivation, and extracting bacterial DNA from the other part of bacteria solution; and obtaining 1,336 lactobacilli in total by virtue of carrying out bacteria 16S rRNA gene amplification and sequencing, carrying out BLAST comparison for sequencing results, and then analyzing and respectively deposited species according to comparison results. The foregoing corresponding lactobacilli are screened by screening low-pH growth tolerance test for the foregoing preserved 1,336 lactobacilli, 16S rRNA genotype screening, test for inhibition of Gardnerella vaginalis, lactic acid production test, hydrogen peroxide production test and test for adhesion on Hela cell test.

A method for screening the lactobacilli provided by the present disclosure includes the following steps:
1. Low-pH growth tolerance test
1.1 Activation: activating the deposited lactobacilli in the MRS broth with a pH value of 6.5, and cultivating overnight at 37° C.;
1.2 Transferring: transferring an activated bacteria solution to the MRS broth with a pH value of 4-5, and measuring an OD₆₀₀ value once every 2-3 h;
1.3 Comparison of the OD₆₀₀ value: screening strains from different samples which can proliferate quickly or has the high OD₆₀₀ value.
2. 16S rRNA genotype screening

If the lactobacilli of the same species separated from the same sample have different 16S rRNA sequences, it is shown that their genotypes are different, and their physiological properties may be also different. Hence, the 16S rRNA genotype screening is carried out for the lactobacilli screened by the comparison of low-pH culture activities.
3. Test for inhibition on Gardnerella vaginalis

After the lactobacilli are activated, taking 0.1 mL of bacteria solution to mix uniformly with the MRS solid medium, pouring into a 6 cm plate, cultivating for 48 h at 37° C. upon complete coagulation, taking out from the plate, and using a puncher with an inner diameter of 6 mm to punch an agar medium, so as to obtain bacteria cakes; and after the Gardnerella vaginalis is activated and transferred, diluting a Gardnerella solution to 100 times with the anaerobic sterile PBS buffer solution by ten consecutive gradients, respectively taking 0.5 mL of 10⁻¹ and 10⁻² diluents and 5.25 mL of BHI solid medium containing 5% horse serum for uniformly mixing, pouring into the 9 cm plate, slightly placing lactobacilli cakes on a BHI agar surface upon complete coagulation, symmetrically placing 4 cakes on every plate in the form of two in parallel, placing into an anaerobic seal pot, adding an anaeropack, forwardly placing the plate for 48 h-culture, and measuring a size of an inhibition zone with a vernier caliper.
4. Lactic acid production test

After the lactobacilli are activated, transferring to the MRS broth medium in the form of two in parallel, cultivating for 48 h at 37° C., determining by a test paper with a pH value of 0.5-5.0, recording the pH value of the lactobacillus solution after the 48 h-culture, and selecting the strains for liquid chromatography by the following two conditions: Condition 1, the liquid chromatography is conducted for the strain with a pH value of 2.5; Condition 2, the strains with low pH values are selected from the same species of lactobacilli for liquid chromatography; after a liquid chromatography sample is determined, diluting a supernatant by 5 times, adding a concentrated sulfuric acid for pretreatment, and filtrating with a 0.22 μm needle filter before sampling. The related liquid chromatography parameters are as follows:
Model of instrument: Agilent, analytical liquid chromatograph 1200
Model of chromatographic column: Bio-Rad, Aminex™ HPX-87H
Mobile phase: 0.005 M H₂SO₄, at the speed of 0.6 mL/min
Detector and detection wave length: DAD, 207 nm; RID, differential refraction signal
Sample amount: 20 μL.
   5. Hydrogen peroxide production capacity

After the lactobacilli are activated, using a pipettor to suck 2 μL of bacteria solution to dibble into a MRS agar containing 0.25 mg/mL of 3,3′,5,5′-tetramethyl benzidine solution and 0.01 mg/mL of horseradish peroxidase, providing two parallel plates at the observation time points of 24 h, 48 h and 72 h respectively, placing the plates at the same observation time point into the same anaerobic seal pot, putting into the anaeropack, cultivating at 37° C., taking out from the corresponding plates for exposure in the air upon the expiry of the corresponding culture time, observing a chromogenic reaction upon 30 min and photographing for recording; and taking Lactobacillus delbrueckii as a positive control, marking 4 points for those deeper than the blue produced by the Lactobacillus delbrueckii, 3 points for those equivalent to the blue produced by the Lactobacillus delbrueckii, 2 points for those shallower than the blue produced by the Lactobacillus delbrueckii, 1 point for those in very light blue (slight chromogenic reaction), and 0 points for non-discolouring.
6. Test for adhesion on Hela cells

After the lactobacilli are activated, centrifugally washing lactobacillus bodies twice, re-suspending with a PBS; sucking 100 μL of lactobacillus suspension into a 96-well cell culture plate containing the Hela cells; standing at 37° C. for 30-min incubation; washing twice with a sterile PBS to wash away non-adhesion lactobacilli; adding 25 μL of pancreatin solution into every well, and placing to a 37° C. incubator to digest cells; after the Hela cells are digested and turned into balls, adding 75 μL of complete medium into each well, repeatedly blowing and beating uniformly; sucking 20 μL of bacterial suspension after complete digesting, diluting with the sterile PBS by ten consecutive gradients, selecting the proper diluting gradient for a pouring-process counting experiment, and counting after cultivating at 37° C. for 48 h.

The ingredients and preparation methods of the above and following bacteria mediums are as follows:
Preparation of a MRS broth: weighing 52.0 g of medium powder of a MRS finished product, and dissolving into 1 L of distilled water; heating and boiling, adding 0.55 g of cysteine hydrochloride after cooling to room temperature, and adjusting a pH value to 6.5 after stirring to dissolve; and installing a quantitative dispenser, feeding N₂, heating until boiling, boiling for 20 min in a micro-boiling state, filling into 10 mL of anaerobic tubes after cooling, carrying out moist heat sterilization for 20 min at a high temperature of 118° C., and storing for further use in shade and away from light.
Preparation of the MRS solid medium: weighing 52.0 g of medium powder of the MRS finished product and 15.0 g of agar powder, dissolving into 1 L of distilled water, heating until boiling, adding 0.55 g of cysteine hydrochloride to adjust the pH value to 6.5 after boiling, carrying out moist heat sterilization for 20 min at the high temperature of 118° C., and storing for further use in shade and away from light. Preparation of a semi-quantitative hydrogen peroxide medium: weighing 52.0 g of medium powder of the MRS finished product and 15.0 g of agar powder, dissolving into 1 L of distilled water, adjusting the pH value to 6.5, carrying out moist heat sterilization for 20 min at the high temperature of 118° C., placing into a 50° C. water bath kettle to insulate for 30 min after sterilization, and adding TMB (a final concentration of the TMB is 0.25 mg/mL) and HRP (a final concentration of the HRP is 0.01 mg/mL) for uniformly mixing; and after cooling and coagulation, marking a culture name and a preparation date, and placing into a 4° C. refrigerator for further use.
Preparation of an anaerobic PBS: weighing 0.27 g of monopotassium phosphate, 1.42 g of disodium hydrogen phosphate, 8 g of sodium chloride and 0.2 g of potassium chloride, dissolving into 1 L of distilled water, heating until boiling, adding 0.55 g of cysteine hydrochloride after cooling to room temperature, adjusting the pH value to 6.5 after stirring and dissolving, installing the quantitative dispenser and feeding N₂, heating until boiling, boiling for 30 min in a micro-boiling state, filling to 10 mL anaerobic tubes after cooling, carrying out moist heat sterilization for 30 min at a high temperature of 121° C., and storing for further use in shade and away from light.
Preparation of an anaerobic BHI liquid medium: weighing 37.0 g of medium powder of a BHI finished product, dissolving into 1 L of distilled water, heating until boiling, adding 0.55 g of cysteine hydrochloride after cooling to room temperature, adjusting the pH value to 6.5 after stirring and dissolving, installing the quantitative dispenser and feeding N₂, heating until boiling, boiling for 20 min in a micro-boiling state, feeding N₂ and CO₂ (a ratio of 1:1) in the course of cooling and filling, filling to 10 mL anaerobic tubes after cooling, carrying out moist heat sterilization for 20 min at a high temperature of 118° C., and storing for further use in shade and away from light.
Preparation of an anaerobic BHI semi-solid medium: weighing 37.0 g of medium powder of the BHI finished product, and dissolving into 1 L of distilled water; heating until boiling, adding 6 g of agar powder and 0.55 g of cysteine hydrochloride after cooling to room temperature, adjusting the pH value to 6.5 after stirring and dissolving, installing the quantitative dispenser and feeding N₂, heating until boiling, boiling for 20 min in the micro-boiling state, feeding N₂ and CO₂ (a ratio of 1:1) in the course of cooling and filling, filling to 10 mL anaerobic tubes in time, carrying out moist heat sterilization for 20 min at a high temperature of 118° C., and storing in shade and away from light.
Preparation of a nutrient broth liquid medium: weighing 10 g of peptone, 3 g of beef powder, 5 g of sodium chloride, dissolving into 1 L of distilled water, adjusting a pH value to 7.2, heating until boiling, and dispensing after cooling to room temperature, 10 mL for each one; and carrying out moist heat sterilization for 15 min at a high temperature of 121° C., and storing in shade and away from light.
Preparation of a nutrient broth solid medium: weighing 10 g of peptone, 3 g of beef powder, 5 g of sodium chloride and 6 g of agar powder, dissolving into 1 L of distilled water, adjusting the pH to 7.2, slightly cooling, and filling into the 10 mL anaerobic tubes in time; and carrying out moist heat sterilization for 15 min at a high temperature of 121° C., and storing in shade and away from light.

For convenient description, the following Lactobacillus johnsonii Ljohn-1 (Deposition No.: CCTCC No.2019426) is hereinafter referred to as “A”; the Lactobacillus crispatus Lcris-2 (Deposition No.: CCTCC No.2019427) is hereinafter referred to as “B”; the Lactobacillus rhamnosus Lrham-6 (Deposition No.: CCTCC No.2019428) is hereinafter referred to as “C”; the Lactobacillus jensenii Ljen-10 (Deposition No.: CCTCC No.2019429) is hereinafter referred to as “D”; and the Lactobacillus gasseri Lgass-17 (Deposition No.: CCTCC No.2019430) is hereinafter referred to as “E”.

Embodiment 1: Comparison of lactic acid production capacities of single and combined lactobacilli.

Single lactobacilli: After the lactobacilli are activated, transfer to a MRS broth medium in the form of two in parallel, cultivate for 48 h at 37° C., determine by a test paper with a pH value of 0.5-5.0, record a pH value of a lactobacillus solution after the 48 h culture, carry out liquid chromatography for strains, dilute a supernatant by 5 times, add a concentrated sulfuric acid for pretreatment, and filtrate with a 0.22 pum syringe-driven filter before sampling.

Combined lactobacilli: After the lactobacilli are activated and transferred, select three or more of strains for mixed culture in the form of three in parallel, cultivate for 48 h at 37° C., centrifuge, dilute the supernatant by 5 times, add the concentrated sulfuric acid for pretreatment, and filtrate with the 0.22 μm syringe-driven filter before sampling. The related liquid chromatography parameters are as follows:
Model of instrument: Agilent, analytical liquid chromatograph 1200
Model of chromatographic column: Bio-Rad, Aminex™ HPX-87H
Mobile phase: 0.005 M H₂SO₄, at the speed of 0.6 mL/min
Detector and detection wave length: DAD, 207 nm; RID, differential refraction signal
Sample amount: 20 μL.

FIGS. 2A and 2B are diagrams that show comparison of lactic acid production capacities of single bacteria and combined bacteria.

Embodiment 2: Comparison of hydrogen peroxide production capacities of single and combined lactobacilli.

Single lactobacilli: After the lactobacilli are activated, use a pipettor to suck 2 μL of bacteria solution to dibble into a MRS agar containing 0.25 mg/mL of 3,3′,5,5′-tetramethyl benzidine solution and 0.01 mg/mL of horseradish peroxidase, provide two parallel plates at the observation time points of 24 h, 48 h and 72 h respectively, place the plates at the same observation time point into the same anaerobic seal pot, cultivate at 37° C., take out from the corresponding plates for exposure in the air upon the expiry of the corresponding culture time, observe a chromogenic reaction upon 30 min and photographing for recording: taking Lactobacillus delbrueckii as a positive control, marking 4 points for those deeper than the blue produced by the Lactobacillus delbrueckii, 3 points for those equivalent to the blue produced by the Lactobacillus delbrueckii, 2 points for those shallower than the blue produced by the Lactobacillus delbrueckii, 1 point for those in very light blue (slight chromogenic reaction), and 0 points for non-discolouring.

Combined lactobacilli: After the lactobacilli are activated and transferred, select three or more strains for mixing, use the pipettor to suck 2 μL of bacteria solution to dibble into the MRS agar containing TMB and HRP, cultivate at 37° C., take out from the corresponding plates for exposure in the air upon the expiry of the corresponding culture time, observe a chromogenic reaction upon 30 min and photograph for recording: taking Lactobacillus delbrueckii as the positive control, marking 4 points for those deeper than the blue produced by the Lactobacillus delbrueckii, 3 points for those equivalent to the blue produced by the Lactobacillus delbrueckii, 2 points for those shallower than the blue produced by the Lactobacillus delbrueckii, 1 point for those in very light blue (slight chromogenic reaction), and 0 points for non-discolouring.

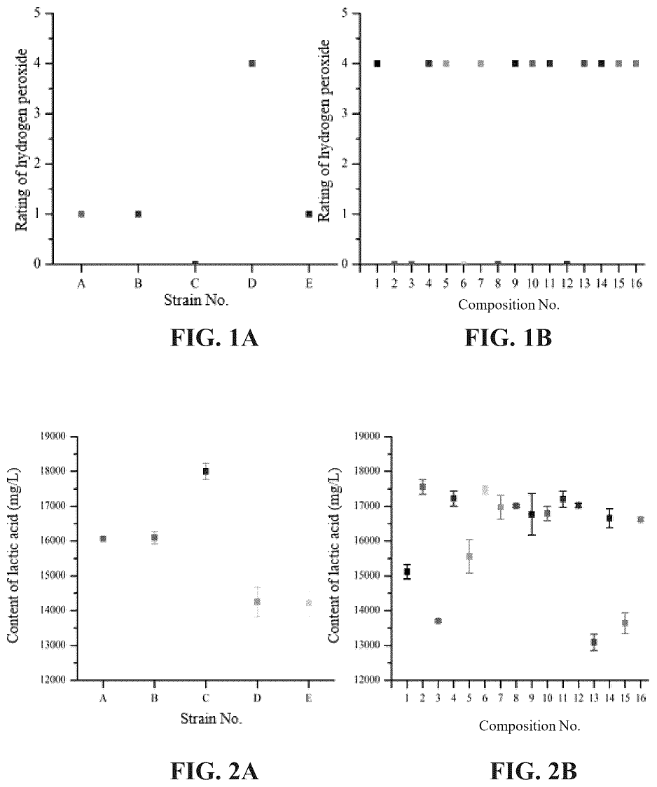

FIGS. 1A and 1B are diagrams that show comparison of rating of hydrogen peroxide production capacities of single bacteria and combined bacteria.

Embodiment 3: Comparison of Gardnerella vaginalis inhibitions of single and combined lactobacilli.

Single lactobacilli: After the lactobacilli are activated, take 0.1 mL of bacteria solution to mix uniformly with the MRS solid medium, pour into a 6 cm plate, cultivate for 48 h at 37° C. upon complete coagulation, take out from the plate, and use a puncher with an inner diameter of 6 mm to punch an agar medium, so as to obtain bacteria cakes; and after the Gardnerella vaginalis is activated and transferred, dilute a gardnerella solution to 100 times with the anaerobic sterile PBS buffer solution by ten consecutive gradients, respectively take 0.5 mL of 10⁻¹ and 10⁻² diluents and 5.25 mL of BHI solid medium containing 5% horse serum for uniformly mixing, pour into the 9 cm plate, slightly place lactobacillus cakes on a BHI agar surface upon complete coagulation, symmetrically place 4 bacteria cakes on every plate in the form of two in parallel, place into an anaerobic seal pot, add an anaeropack, forwardly place the plate to cultivate for 48 h, and measure a size of an inhibition zone with a vernier caliper.

Combined lactobacilli: After the lactobacilli are activated and transferred, select three or more strains for mixing, take 0.1 mL of bacteria solution to mix uniformly with the MRS solid medium, pour into a 6 cm plate, cultivate for 48 h at 37° C. upon complete coagulation, take out from the plate, and use a puncher with the inner diameter of 6 mm to punch the agar medium, so as to obtain the bacteria cake; and after the Gardnerella vaginalis is activated and transferred, dilute a gardnerella solution to 100 times with the anaerobic sterile PBS buffer solution by ten consecutive gradients, respectively take 0.5 mL of 10-1 and 10-2 diluents and 5.25 mL of BHI solid medium containing 5% horse serum for uniformly mixing, pour into the 9 cm plate, slightly place lactobacillus cakes on a BHI agar surface upon complete coagulation, symmetrically place 4 bacteria cakes on every plate in the form of two in parallel, place into an anaerobic seal pot, add an anaeropack, forwardly place the plate to cultivate for 48 h, and measure a size of an inhibition zone with a vernier caliper.

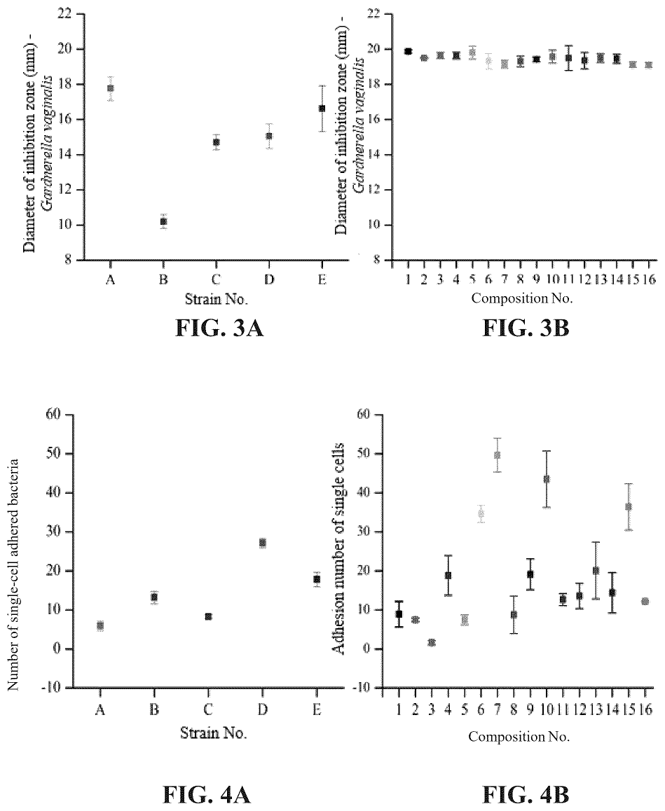

FIGS. 3A and 3B are diagrams that show comparison of inhibitions on Gardnerella vaginalis by single bacteria and combined bacteria.

Embodiment 4: Comparison of adhesion on hela cells by single bacteria and combined bacteria.

Single bacteria: After the lactobacilli are activated, centrifugally wash lactobacillus bodies twice, and re-suspend with a PBS; suck 100 μL of lactobacillus suspension into a 96-well cell culture plate containing Hela cells, stand at 37° C. for incubation for 30 min, and wash twice with the sterile PBS after 30 min to wash away non-adhesion lactobacilli; add 25 μL of pancreatin solution into every well of the 96-well cell culture plate containing the Hela cells, and place to a 37° C. incubator to digest cells; after the Hela cells are digested and turned into balls, add 75 μL of complete medium into each well, and repeatedly blow and beat uniformly; suck 20 μL of bacterial suspension after complete digesting, dilute with the sterile PBS by ten consecutive gradients, select the proper diluting gradient for a pouring-process counting experiment, and count after cultivating at 37° C. for 48 h.

Combined bacteria: After the lactobacilli are activated and transferred, centrifugally wash bacteria bodies twice, re-suspend with a PBS, select bacteria solution of three or more strains for mixing uniformly, suck 100 μL of lactobacillus suspension into the 96-well cell culture plate containing the Hela cells, stand at 37° C. for incubation for 30 min and 4 h, and wash with the sterile PBS twice upon 30 min and 4 h to wash away non-adhered lactobacilli; upon expiry of the corresponding incubation time, add 25 μL of pancreatin solution into each well of the 96-well cell culture plate containing the Hela cells, and place into the 37° C. incubator to digest cells; after the Hela cells are digested and turned into balls, add 75 μL of complete medium into each well, and repeatedly blow and beat uniformly; suck 20 μL of bacterial suspension after complete digesting, dilute with the sterile PBS by ten consecutive gradients, select the 10⁻⁴ diluting gradient for a pouring-process counting experiment, and count after cultivating at 37° C. for 48 h.

FIGS. 4A and 4B are diagrams that show comparison of adhesions on hela cells by single bacteria and combined bacteria.

Embodiment 5: Comparison of inhibitions on Staphylococcus aureus, Pseudomonas aeruginosa, Escherichia coli, Salmonella paratyphi B and Shigella dysenteriae by single bacteria and combined bacteria.

Single lactobacilli: After the lactobacilli are activated, take 0.1 mL of bacteria solution to mix uniformly with the MRS solid medium, pour into a 6 cm plate, cultivate for 48 h at 37° C. upon complete coagulation, take out from the plate, and use a puncher with an inner diameter of 6 mm to punch an agar medium, so as to obtain bacteria cakes; and After the Staphylococcus aureus, the Pseudomonas aeruginosa, the Escherichia coli, the Salmonella paratyphi B and the Shigella dysenteriae are activated and transferred, dilute a pathogenic bacteria solution to 100 times with 0.9% normal saline (NS) by ten consecutive gradients, respectively take 0.5 mL of 10⁻¹ and 10⁻² diluents and 5 mL of nutrient agar solid medium for uniformly mixing, pour into a 9 cm plate, slightly place lactobacilli cakes on a nutrient agar surface upon complete coagulation, symmetrically place 4 cakes on every plate in the form of two in parallel, place into a seal pot, forwardly place the plate to cultivate for 24 h, and measure a size of an inhibition zone with a vernier caliper.

Combined bacteria: Inhibition on Salmonella paratyphi B and Shigella dysenteriae After the lactobacilli are activated and transferred, select three or more strains for mixing, take 0.1 mL of bacteria solution to mix uniformly with a MRS solid medium, pour into a 6 cm plate, cultivate for 48 h at 37° C. upon complete coagulation, take out from the plate, and use a puncher with an inner diameter of 6 mm to punch an agar medium, so as to obtain bacteria cakes; and after the Staphylococcus aureus, the Staphylococcus aureus, the Pseudomonas aeruginosa, the Escherichia coli, the Salmonella paratyphi B and the Shigella dysenteriae are activated and transferred, dilute the pathogenic bacteria solution to 100 times with 0.9% normal saline (NS) by ten consecutive gradients, respectively take 0.5 mL of 10⁻¹ and 10⁻² diluents and 5 mL of nutrient agar solid medium for uniformly mixing, pour into a 9 cm plate, slightly place lactobacilli cakes on a nutrient agar surface upon complete coagulation, symmetrically place 4 cakes on every plate in the form of two in parallel, place into a seal pot, forwardly place the plate to cultivate for 24 h, and measure a size of an inhibition zone with a vernier caliper.

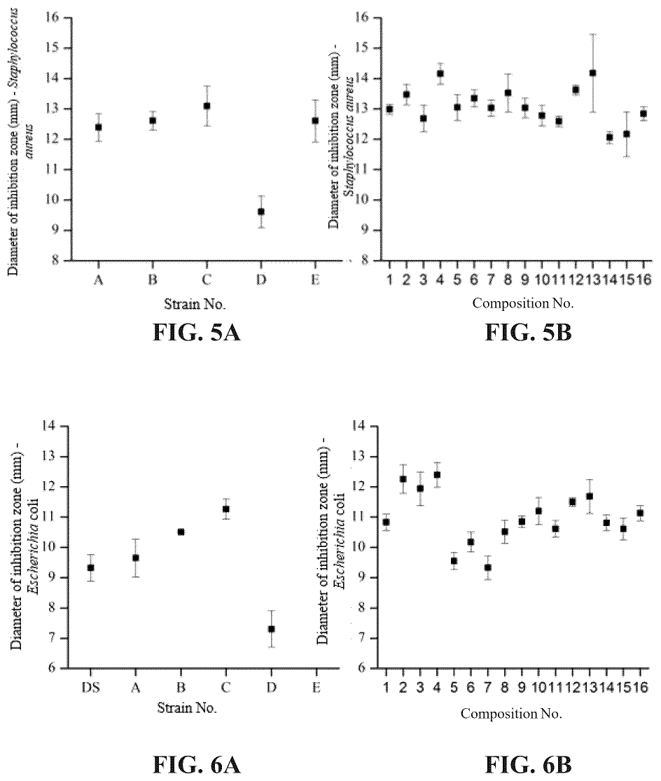

FIGS. 5A and 5B are diagrams that show comparison of inhibitions on Staphylococcus aureus by single bacteria and combined bacteria.

FIGS. 6A and 6B are diagrams that show comparison of inhibitions on Escherichia coli by single bacteria and combined bacteria.

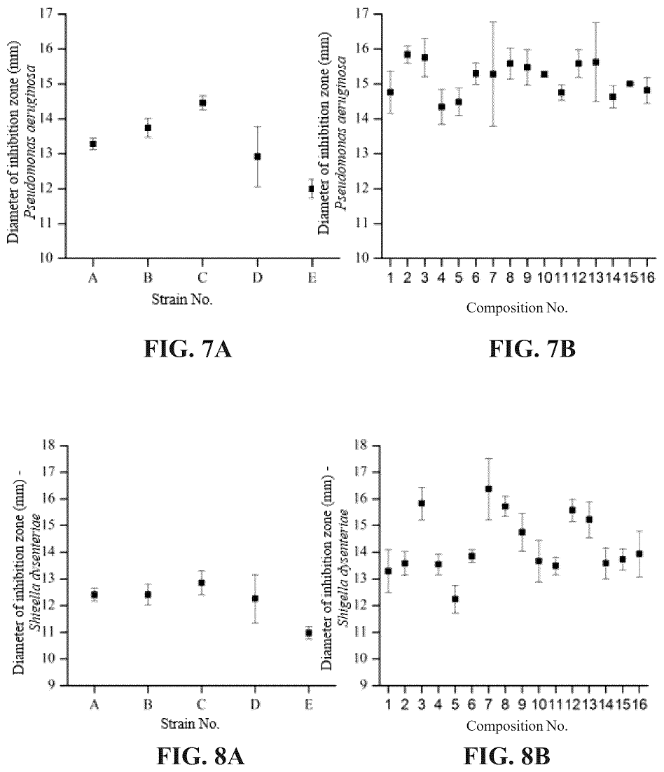

FIGS. 7A and 7B are diagrams that show comparison of inhibitions on Pseudomonas aeruginosa by single bacteria and combined bacteria.

FIGS. 8A and 8B are diagrams that show comparison of inhibitions on Shigella dysenteriae by single bacteria and combined bacteria.

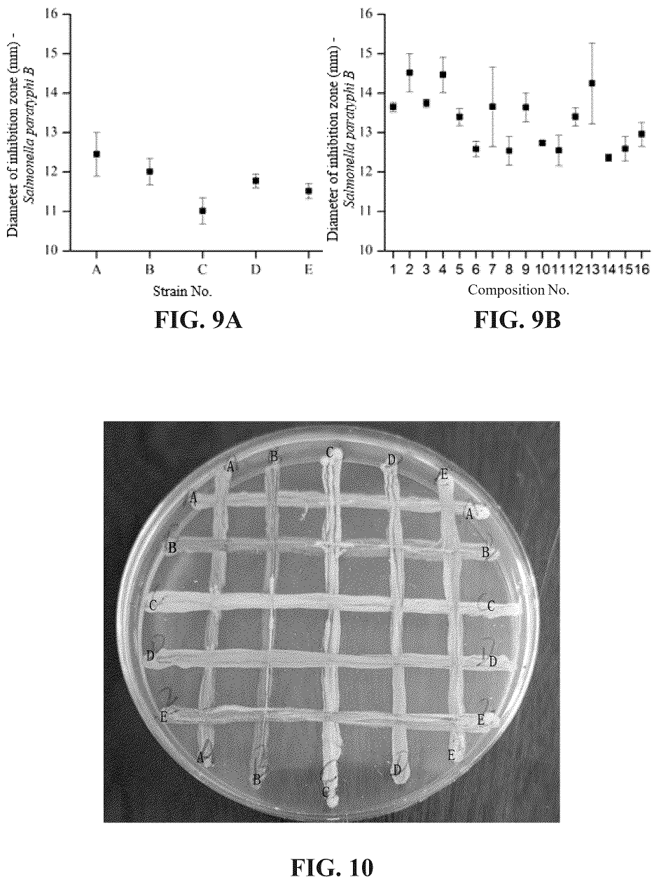

FIGS. 9A and 9B are diagrams that show comparison of inhibitions on Salmonella paratyphi B by single bacteria and combined bacteria.

According to comparison experiments in the foregoing Embodiments 1-5, the comparison results are shown in Table 6 below:

TABLE 6
Comparison of probiotic abilities of single bacteria and combined bacteria

| | Microbiological composition | | | | |
|---|---|---|---|---|---|
| | (number before the letter | | | | |
| | expresses content ratio of | Diameteter of | | | |
| Microorganism | single strain corresponding | inhibition zone | Rating of hydrogen | Content of lactic | Adhesion number |
| description | to the letter) | of Gardnerella (mm) | peroxide capacity | acid (mg/L) | of single cells |
| DS | Lactobacillus delbrueckii | 14.29 | 3 | 12964.12 | 7.7 |
| A | A | 17.77 | 1 | 16064.53 | 5.95 |
| B | B | 10.19 | 1 | 16098.44 | 13.2 |
| C | C | 14.71 | 0 | 18001.19 | 8.3 |
| D | D | 15.06 | 4 | 14258.47 | 27.15 |
| E | E | 16.63 | 1 | 14219.11 | 17.8 |
| 1 | 1A + 1B + 1D | 19.87 | 4 | 15117.36 | 8.91 |
| 2 | 1A + 1B + 1C | 19.50 | 0 | 17555.32 | 7.5 |
| 3 | 1A + 1B + 1E | 19.65 | 0 | 13703.73 | 1.64 |
| 4 | 1B + 1C + 1D | 19.65 | 4 | 17224.19 | 18.83 |
| 5 | 1B + 1D + 1E | 19.81 | 4 | 15562.84 | 7.5 |
| 6 | 1B + 1C + 1E | 19.34 | 0 | 17479.38 | 34.67 |
| 7 | 1A + 1C + 1D | 19.16 | 4 | 16974.68 | 49.68 |
| 8 | 1A + 1C + 1E | 19.31 | 0 | 17012.27 | 8.75 |
| 9 | 1A + 1D + 1E | 19.42 | 4 | 16769.17 | 19.14 |
| 10 | 1C + 1D + 1E | 19.58 | 4 | 16794.06 | 43.5 |
| 11 | 1A + 1B + 1C + 1D | 19.50 | 4 | 17203.57 | 12.67 |
| 12 | 1A + 1B + 1C + 1E | 19.35 | 0 | 17023.87 | 13.58 |
| 13 | 1A + 1B + 1D + 1E | 19.50 | 4 | 13091.07 | 20.08 |
| 14 | 1B + 1C + 1D + 1E | 19.46 | 4 | 16653.29 | 14.42 |
| 15 | 1A + 1C + 1D + 1E | 19.13 | 4 | 13642.85 | 36.42 |
| 16 | 1A + 1B + 1C + 1D + 1E | 19.11 | 4 | 16618.79 | 12.17 |
| | Diameter of | | | Diameter of | Diameter of |
| | inhibition zone of | Diameter of | Diameter of | inhibition zone of | inhibition zone of |
| Microorganism | Staphylococcus | inhibition zone of | inhibition zone of | Psuedomonas | Salmonella |
| description | aureus (mm) | Escherichia coli (mm) | Shigella dysenteriae (mm) | aeruginosa (mm) | paratyphi B (mm) |
| DS | 11.28 | 9.32 | 11.93 | 13.09 | 12.00 |
| A | 12.39 | 9.65 | 12.41 | 13.28 | 12.45 |
| B | 12.61 | 10.51 | 12.41 | 13.74 | 12.01 |
| C | 13.09 | 11.26 | 12.85 | 14.46 | 11.01 |
| D | 9.61 | 7.30 | 12.26 | 12.92 | 11.78 |
| E | 12.61 | 9.29 | 10.98 | 12.00 | 11.52 |
| 1 | 12.99 | 10.83 | 13.29 | 14.76 | 13.65 |
| 2 | 13.47 | 12.26 | 13.58 | 15.84 | 14.52 |
| 3 | 12.68 | 11.94 | 15.83 | 15.76 | 13.75 |
| 4 | 14.16 | 12.40 | 13.55 | 14.34 | 14.47 |
| 5 | 13.05 | 9.55 | 12.24 | 14.49 | 13.40 |
| 6 | 13.35 | 10.17 | 13.85 | 15.30 | 12.58 |
| 7 | 13.03 | 9.33 | 16.37 | 15.28 | 13.66 |
| 8 | 13.53 | 10.52 | 15.72 | 15.59 | 12.54 |
| 9 | 13.03 | 10.85 | 14.75 | 15.48 | 13.64 |
| 10 | 12.78 | 11.20 | 13.67 | 15.28 | 12.74 |
| 11 | 12.59 | 10.61 | 13.49 | 14.75 | 12.55 |
| 12 | 13.62 | 11.50 | 15.58 | 15.59 | 13.40 |
| 13 | 14.18 | 11.68 | 15.23 | 15.62 | 14.25 |
| 14 | 12.06 | 10.81 | 13.59 | 14.63 | 12.36 |
| 15 | 12.17 | 10.61 | 13.73 | 15.01 | 12.59 |
| 16 | 12.84 | 11.13 | 13.94 | 14.82 | 12.96 |

Embodiment 6: Test for antagonistic and symbiotic effects among all strains in the multi-lactobacillus.

Use one of strains to be determined to draw a straight line on a medium plate, transversely inoculate two of strains to be determined at both sides of the one of strains to be determined, cultivate for 18-24 h in a cross manner, and determine a bacteriostatic width of a cross point.

Though the tests, the results of antagonistic and symbiotic effects among all strains (bacteriostatic semi-diameter: mm) are shown in Table 7:

TABLE 7
Diameters (mm) of Symbiotic Inhibition Zones of
All Single Bacteria on Solid Plate

| | A | B | C | D | E |
|---|---|---|---|---|---|
| A | 0.00 | 0.00 | 0.00 | 0.00 | 0.00 |
| B | 0.00 | 0.00 | 0.00 | 0.00 | 0.00 |
| C | 0.00 | 0.00 | 0.00 | 0.00 | 0.00 |
| D | 0.00 | 0.00 | 0.00 | 0.00 | 0.00 |
| E | 0.00 | 0.00 | 0.00 | 0.00 | 0.00 |

Inoculate the single strain to be determined to a MRS broth, cultivate at 37° C. for 8 h, dilute the single strain to a proper gradient by ten consecutive gradients before inoculation, count a concentration of the bacteria solution before inoculation in such a manner of three in parallel at every gradient, place the plate into an anaerobic seal pot, put an anaeropack, and cultivate at 37° C. for 48 h; inoculate the strains to be determined to the MRS broth after mixing, wherein an inoculation concentration of each bacterium in the mixed bacteria is the same as an inoculation concentration of the single strain, and cultivate at 37° C. for 8 h; dilute the single and mixed bacteria MRS broths, after being cultivated for about 8 h, to the proper gradient by ten consecutive gradients, coat and count in such a manner of three parallel at every gradient, place the plate into the anaerobic seal pot, put an anaeropack, and cultivate at 37° C. for 48 h; after the plate is cultivated for 48 h anaerobically, calculate bacterial colonies before and after inoculation for the single bacteria plate, and analyze amplification multiples of the single bacteria; and meanwhile, carry out mass spectrum identification for the flora on the mixed bacteria plate, determine species through a mass spectrum to analyze the amplification multiple of the single bacteria in the mixed bacteria, and prove no inhibition among the strains but they may grow together through the tests, see Table 8 for broth co-culture amplification data.

TABLE 8
Amplification Multiples of Symbiotic Single Bacteria on MRS Broth

| | Amplification | Amplification | |
|---|---|---|---|
| | multiples | multiples | Amplification |
| | calculated by | calculated by | multiples |
| | mixed culture | mixed culture | calculated |
| Strain | mass spectrum- | mass spectrum- | by single |
| No. | parallel 1 | parallel 2 | bacteria culture |
| A | 14.73 | 10.85 | 17.17 |
| B | 9.74 | 12.82 | 17.57 |
| C | 85.08 | 81.73 | 92.15 |
| D | 38.22 | 51.4 | 29.06 |
| E | 75.19 | 71.3 | 103.69 |

FIG. 10 is a test diagram 1 of a solid symbiotic effect between all single bacteria in multi-lactobacillus.

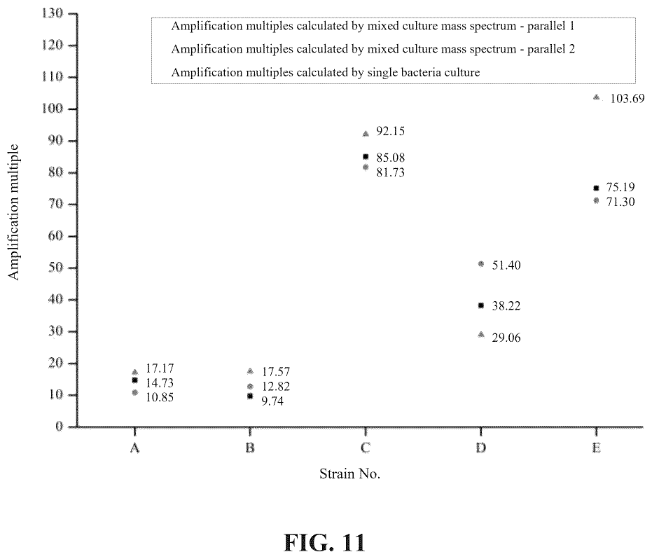

FIG. 11 is a test diagram 2 of a symbiotic effect of a broth between all single bacteria in multi-lactobacillus.

### (II) Preparation of Live Bacteria Capsules

The following four strains of bacteria were selected to prepare live bacteria capsules: Lactobacillus johnsonii Ljohn-1, Lactobacillus crispatus Lcris-2, Lactobacillus jensenii Ljen-10, and Lactobacillus gasseri Lgass-17.

Based on CFU, in each unit formulation of the microbial formulation of the present disclosure, any two of the bacteria were in an amount ratio of (1-100):(1-100). In each unit formulation, the total viable count of the bacteria as the active ingredients was not less than 1×10⁷ CFU, and the viable count of each of the strains was not less than 1×10⁶ CFU.

The fermentation and cultivation of the strains, the lyophilization, the preparation of bacterial powder, and the preparation of capsules were completed using conventional techniques in the art. For example, reference may be made to: Principles of Fermentation Engineering; Fermentation Engineering Protocols; Principles and Technology Applications of Fermentation Engineering; Microbial Cultivation Techniques; Industrial Strain Preparation Technology; Principles of Fermentation Technology; Lactic Acid Bacteria: Microbiology and Functional Aspects; Encyclopedia of Bioprocess Technology: Fermentation, Biocatalysis, and Bioseparation; Bacteriological Analytical Manual Online; Practical Techniques for Pharmaceutical Packaging; and the like. Especially, reference may be made to the methods described in patents CN117165438A and CN117165439A.
(1) Preparation Example 1 for Microbial Formulation (Live Bacteria Capsule)

The 4 strains were each subjected to seed solution cultivation, fermentation culture, bacterial cell collection by centrifugation, bacterial cell washing, and secondary bacterial cell collection by centrifugation to obtain bacterial sludge. Then, the bacterial sludge was mixed with a lyoprotectant solution in a mass ratio of 1:1, followed by lyophilization, pulverization, and sieving through a 40-mesh sieve to prepare bacterial powder (undersized product). The components of the lyoprotectant solution were as follows: 40% of trehalose, 0.3% of xanthan gum, 5% of fructooligosaccharide, 5% of sodium glutamate, 5% of sodium ascorbate, and the balance of purified water (based on mass percentage).

The resulting bacterial powders were added at 2.5×10⁶ CFU per strain per unit formulation and then supplemented with the excipient lactose to achieve a specification of 0.16 g per unit formulation. The mixture was uniformly mixed and filled into No. 3 gelatin capsules. The capsules were then packaged with double aluminum blister packaging.
(2) Preparation Example 2 for Microbial Formulation (Live Bacteria Capsule)

The 4 strains were each subjected to seed solution cultivation, fermentation culture, bacterial cell collection by centrifugation, bacterial cell washing, and secondary bacterial cell collection by centrifugation to obtain bacterial sludge. Then, the bacterial sludge was mixed with a lyoprotectant solution in a mass ratio of 1:2, followed by lyophilization, pulverization, and sieving through a 40-mesh sieve to prepare bacterial powder (undersized product). The components of the lyoprotectant solution were as follows: 40% of trehalose, 0.3% of xanthan gum, 5% of fructooligosaccharide, 5% of sodium glutamate, 5% of sodium ascorbate, and the balance of purified water (based on mass percentage).

The resulting bacterial powders were added at 2.5×10¹⁰ CFU per strain per unit formulation and then supplemented with the excipient lactose to achieve a specification of 0.16 g per unit formulation. The mixture was uniformly mixed and filled into No. 3 gelatin capsules. The capsules were then packaged with double aluminum blister packaging.
(3) Preparation Example 3 for Microbial Formulation (Live Bacteria Capsule)

The 4 strains were each subjected to seed solution cultivation, fermentation culture, bacterial cell collection by centrifugation, bacterial cell washing, and secondary bacterial cell collection by centrifugation to obtain bacterial sludge. Then, the bacterial sludge was mixed with a lyoprotectant solution in a mass ratio of 1:5, followed by lyophilization, pulverization, and sieving through a 40-mesh sieve to prepare bacterial powder (undersized product). The components of the lyoprotectant solution were as follows: 40% of trehalose, 0.3% of xanthan gum, 5% of fructooligosaccharide, 5% of sodium glutamate, 5% of sodium ascorbate, and the balance of purified water (based on mass percentage).

The resulting bacterial powders were added at 5×10¹² CFU per strain per unit formulation and then supplemented with the excipient lactose to achieve a specification of 0.16 g per unit formulation. The mixture was uniformly mixed and filled into No. 3 gelatin capsules. The capsules were then packaged with double aluminum blister packaging.
(4) Preparation Example 4 for Microbial Formulation (Live Bacteria Capsule)

The 4 strains were each subjected to seed solution cultivation, fermentation culture, bacterial cell collection by centrifugation, bacterial cell washing, and secondary bacterial cell collection by centrifugation to obtain bacterial sludge. Then, the bacterial sludge was mixed with a lyoprotectant solution in a mass ratio of 1:1, followed by lyophilization, pulverization, and sieving through a 40-mesh sieve to prepare bacterial powder (undersized product). The components of the lyoprotectant solution were as follows: 40% of trehalose, 0.3% of xanthan gum, 5% of fructooligosaccharide, 5% of sodium glutamate, 5% of sodium ascorbate, and the balance of purified water (based on mass percentage).

The resulting bacterial powders were added at 2.5×10⁷ CFU per strain per unit formulation and then supplemented with the excipient lactose to achieve a specification of 0.16 g per unit formulation. The mixture was uniformly mixed and filled into No. 3 gelatin capsules. The capsules were then packaged with double aluminum blister packaging.
(5) Preparation Example 5 for Microbial Formulation (Live Bacteria Capsule)

The 4 strains were each subjected to seed solution cultivation, fermentation culture, bacterial cell collection by centrifugation, bacterial cell washing, and secondary bacterial cell collection by centrifugation to obtain bacterial sludge. Then, the bacterial sludge was mixed with a lyoprotectant solution in a mass ratio of 1:1, followed by lyophilization, pulverization, and sieving through a 40-mesh sieve to prepare bacterial powder (undersized product). The components of the lyoprotectant solution were as follows: 40% of trehalose, 0.3% of xanthan gum, 5% of fructooligosaccharide, 5% of sodium glutamate, 5% of sodium ascorbate, and the balance of purified water (based on mass percentage).

The resulting bacterial powders were added at 2.5×10⁸ CFU per strain per unit formulation and then supplemented with the excipient lactose to achieve a specification of 0.16 g per unit formulation. The mixture was uniformly mixed and filled into No. 3 gelatin capsules. The capsules were then packaged with double aluminum blister packaging.
(6) Preparation Example 6 for Microbial Formulation (Live Bacteria Capsule)

The 4 strains were each subjected to seed solution cultivation, fermentation culture, bacterial cell collection by centrifugation, bacterial cell washing, and secondary bacterial cell collection by centrifugation to obtain bacterial sludge. Then, the bacterial sludge was mixed with a lyoprotectant solution in a mass ratio of 1:1, followed by lyophilization, pulverization, and sieving through a 40-mesh sieve to prepare bacterial powder (undersized product). The components of the lyoprotectant solution were as follows: 40% of trehalose, 0.3% of xanthan gum, 5% of fructooligosaccharide, 5% of sodium glutamate, 5% of sodium ascorbate, and the balance of purified water (based on weight percentage).

The resulting bacterial powders were added at 2.5×10⁹ CFU per strain per unit formulation and then supplemented with the excipient lactose to achieve a specification of 0.16 g per unit formulation. The mixture was uniformly mixed and filled into No. 3 gelatin capsules. The capsules were then packaged with double aluminum blister packaging.

### (III) Preparation of Placebo

The preparation method for the placebo (live bacteria capsule mimic) was as follows:

The excipient microcrystalline cellulose was filled into empty No. 3 gelatin capsules at 0.11 g/capsule. The capsules were then packaged with double aluminum blister packaging.

### Example 2: Phase I Clinical Trial for Safety and Tolerability of Microbial Formulations

A single-center, randomized, double-blind, placebo-controlled, single/multiple-dose, dose-escalating trial design was adopted to evaluate the safety and tolerability in healthy subjects after single/multiple-dose escalation administration of the microbial formulations (live bacteria capsule) of the present disclosure.

The single-dose study employed a single-center, randomized, double-blind, placebo-controlled, dose-escalating trial design.

In this single-dose, dose-escalating study for safety and tolerability, a total of 3 dose groups were set in the experimental group, namely A1 (1.0×10⁸ CFU/capsule, Preparation Example 4), A2 (1.0×10⁹ CFU/capsule, Preparation Example 5), and A3 (1.0×10¹⁰ CFU/capsule, Preparation Example 6) dose groups; the placebo group received the live bacteria capsule mimic. A total of 24 subjects were included in the 3 groups. Details regarding the number of subjects in each group, dose-escalation design, and administration regimen are specified in Table 9 below:

TABLE 9
Administration regimen for subjects in single-dose study

| Group No. | A1 | A2 | A3 |
|---|---|---|---|
| Escalation ratio | — | 10-fold | 10-fold |
| Dose of administration | 1.0 × 10⁸ | 1.0 × 10⁹ | 1.0 × 10¹⁰ |
| (CFU/capsule, 1 capsule) | | | |
| Number of subjects (experimental | 6 + 2 | 6 + 2 | 6 + 2 |
| group + placebo group) | | | |

In the multiple-dose, dose-escalating study for safety and tolerability, a total of 2 dose groups were set, namely B1 (1.0×10⁹ CFU/capsule, Preparation Example 5) and B2 (1.0×10¹⁰ CFU/capsule, Preparation Example 6) dose groups; the placebo groups received the live bacteria capsule mimic. In the multiple-dose study, each subject received only one dose, which was administered vaginally for 7 consecutive days. A total of 20 subjects were included in the 2 groups. Details regarding the number of subjects in each group, dose-escalation design, and administration regimen are specified in Table 10 below:

TABLE 10
Administration regimen for subjects in multiple-dose study

| Group No. | B1 | B2 |
|---|---|---|
| Escalation ratio | — | 10-fold |
| Dose of administration (CFU/capsule, 1 capsule) | 1.0 × 10⁹ | 1.0 × 10¹⁰ |
| Days of administration | 7 days | 7 days |
| Number of subjects (experimental | 8 + 2 | 8 + 2 |
| group + placebo group) | | |

The analysis results showed that there were no significant differences in the incidence of adverse events and adverse reactions between the experimental group and the placebo group in both the single-dose and multiple-dose studies (P>0.05), indicating that the live bacteria capsules were safe and well-tolerated after single-dose and multiple-dose vaginal administrations in healthy subjects.

### Example 3: Phase II Clinical Trial of Microbial Formulation for Treating Atrophic Vaginitis

The phase I clinical study on the safety and tolerability of microbial formulations in healthy subjects showed that the microbial formulations were safe and well-tolerated. Based on this, a phase II study was designed and conducted.

### (I) Design of Trial

This trial was a multicenter, randomized, double-blind, placebo-controlled phase II clinical study aimed at evaluating the efficacy and safety of a microbial formulation (live bacteria capsule, prepared in Preparation Example 5 of Example 1) in treating postmenopausal atrophic vaginitis. The study included three parts: a screening period (D-14 to D-1), a treatment period (Wk 1 to Wk 12), and a follow-up period (Wk 13 to Wk 16).

In this study, postmenopausal female subjects with at least moderate or severe vaginal dryness (for sexually active subjects, this must be accompanied by moderate or severe dyspareunia) were enrolled.

Eligible subjects were randomly assigned to the experimental group or the control group according to a ratio of 2:1 (based on the number of subjects). Four weeks (28 days) constituted 1 treatment cycle. That is, the live bacteria capsule (total viable count of 1×10⁹ CFU/capsule) or the placebo was administered once daily during the first 2 weeks of each treatment cycle, with 1 capsule per administration, and no medication was given during the last 2 weeks of each treatment cycle. The full treatment was performed continuously for 3 cycles (12 weeks). This was followed by a 4-week follow-up period.

TABLE 11
Treatment regimes

| | | Number of |
|---|---|---|
| Group | Treatment | subjects |
| Experimental | The live bacteria capsule was | 80 |
| group | administered vaginally once daily, with 1 | |
| | capsule per administration; 4 weeks | |
| | constituted 1 treatment cycle; | |
| | medication was given during the first | |
| | 2 weeks of each treatment cycle, | |
| | and no medication was given during | |
| | the last 2 weeks of each cycle; a | |
| | total of 3 cycles were completed | |
| Control | The placebo (live bacteria capsule | 40 |
| group | mimic, prepared in Example 1) was | |
| | administered vaginally once daily, | |
| | with 1 capsule per administration; 4 | |
| | weeks constituted 1 treatment cycle; | |
| | medication was given during the | |
| | first 2 weeks of each treatment cycle, | |
| | and no medication was given | |
| | during the last 2 weeks of each cycle; | |
| | a total of 3 cycles were completed | |

During the treatment period, the efficacy of the live bacteria capsule relative to the placebo was evaluated or explored based on microecology scores, vaginal symptom scores, vaginal sign scores, vaginal flora colonization, and QoL (regarding female sexual function and vaginal aging).

The safety of the live bacteria capsule relative to the placebo was evaluated based on AEs, SAEs, clinical laboratory tests, vital signs, physical examinations, gynecological examinations, ECGs, and other examinations. After the end of treatment, extended follow-up was performed to evaluate efficacy and safety.

### (II) Inclusion Criteria for Subjects

Subjects who met all of the following criteria were included in this study: 1. voluntarily signing an informed consent form and being able to comply with protocol procedures; 2. being female with a sexual history, aged≥45 years and ≤70 years at the time of informed consent; 3. at the time of screening, having chief complaint of natural menopause≥12 months or having medical records showing bilateral oophorectomy≥6 months, or having follicle-stimulating hormone (FSH)≥40 IU/L and estradiol (E2)≤20 pg/mL; and 4. within 3 months prior to screening, having suffered from moderate or severe vaginal dryness (for sexually active subjects [having a stable partner and engaging in or attempting to engage in ≥1 vaginal intercourse per month], this must be accompanied by moderate or severe dyspareunia).

Note: Vaginal dryness: moderate—vaginal dryness discomfort is felt most of the time, but it does not affect daily life; severe—vaginal dryness discomfort is felt all the time, affecting daily life. Dyspareunia: moderate—sexual intercourse pain is present most of the time, with minimal satisfaction from intercourse and frequent need to discontinue; severe—sexual intercourse pain is present all the time, with inability to enjoy intercourse, occasional post-coital bleeding, and potential abstinence due to sexual intercourse pain.

From the date of signing the informed consent form, subjects should agree not to perform vaginal douching and not to use vaginal lubricants/humectants or other similar agents or devices.

### (III) Exclusion Criteria

Subjects who met any one or more of the following criteria were excluded from this study: 1. at the time of screening, having a concurrent additional reproductive tract infection such as vulvovaginal candidiasis (VVC), trichomonal vaginitis (TV), etc.; 2. at the time of screening, having concurrent severe aerobic vaginitis (AV, defined as an AV score≥7) or being judged by the investigator to require antibiotic treatment; 3. within 2 weeks prior to screening, having received systemic or vaginal administration of an antimicrobial drug, a sex hormone drug, or a microecological preparation; 4. within 10 weeks prior to screening, having undergone vulvar, vaginal, cervical, or pelvic surgery or surgical procedures; 5.
at the time of screening, having unexplained abnormal vaginal bleeding or a clinically significant gynecological disease, including but not limited to vulvar dystrophy, vulvar eczema, and cervical atypical squamous epithelial cells—not excluding high-grade squamous intraepithelial lesion (ASC-H), uterine prolapse (grade 2 or higher), endometrial hyperplasia, ovarian tumor, etc.; 6. within 5 years prior to screening, having suffered from a malignancy [except for the following: a. cervical carcinoma in situ treated with cervical conization; b. surgically cured basal cell carcinoma, squamous cell carcinoma, and/or carcinoma in situ (Bowen's disease) of the skin]; 7. at the time of screening, being positive for serum anti-human immunodeficiency virus antibody (anti-HIV), being positive for hepatitis B virus surface antigen (HBsAg) with hepatitis B virus deoxyribonucleic acid (HBV DNA)≥1000 IU/mL, being positive for anti-hepatitis C virus antibody (anti-HCV) and hepatitis C virus ribonucleic acid (HCV RNA), or being positive for anti-treponema pallidum antibody (anti-Tp); 8. at the time of screening, having hemoglobin (Hb)<90 g/L, white blood cell count (WBC)<3×10⁹/L or >11×10⁹/L, or platelet count (PLT)<75×10⁹/L; 9. at the time of screening, having alanine aminotransferase (ALT) or aspartate aminotransferase (AST)>2×upper limit of normal (ULN), total bilirubin (TBIL)>1.5×ULN, or serum creatinine (SCr)>1.2×ULN; 10. at the time of screening, being judged by the investigator to have a poorly controlled clinically significant disease (including cardiovascular system, respiratory system, digestive system, endocrine/metabolic system, neuropsychiatric system, hematological system, and immune system diseases, etc.), such as systolic blood pressure≥140 mmHg (systolic blood pressure≥150 mmHg for elderly subjects [≥60 years]) and/or diastolic blood pressure≥90 mmHg under resting conditions on different days, fasting blood glucose≥13.9 mmol/L on different days or random blood glucose≥11.1 mmol/L on different days, or NYHA heart function of class III or IV; 11. at the time of screening, being judged by the investigator to be likely to undergo elective surgery within the next 3 months; 12. having a history of drug abuse (social, psychological, and physiological disorders caused by excessive, incorrect, or addictive use of drugs for any non-medical reason) or alcohol abuse [i.e., drinking more than 14 units of alcohol per week (1 unit=360 mL of beer, or 45 mL of spirits with 40% alcohol content, or 150 mL of wine)] within 5 years prior to screening; 13. having a history of severe allergic reactions to the drug or its other ingredients (e.g., lactose or gelatin) (immediate, life-threatening systemic anaphylaxis); 14. within 3 months prior to screening, having participated in other interventional clinical trials, including drugs, vaccines, or devices (excluding observational trials of marketed drugs); and 15. being judged by the investigator to have other conditions that may interfere with the assessment of safety or efficacy in this trial.

### (IV) Evaluation Criteria

Primary efficacy endpoint:

Change from baseline in vaginal dryness symptom score at week 10 of treatment (for all subjects).

Secondary efficacy endpoints:

Changes from baseline in vaginal dryness symptom score at weeks 2, 4, 8, 12, and 16 of treatment (for all subjects); changes from baseline in dyspareunia symptom score at weeks 2, 4, 8, 10, 12, and 16 of treatment (for sexually active subjects only); changes from baseline in other vulvovaginal symptom scores at weeks 2, 4, 8, 10, 12, and 16 of treatment (for subjects with corresponding symptoms only); changes from baseline in clue cell positivity rate at weeks 2, 4, 8, 10, 12, and 16 of treatment; changes from baseline in Nugent score at weeks 2, 4, 8, 10, 12, and 16 of treatment; changes from baseline in Donders score at weeks 2, 4, 8, 10, 12, and 16 of treatment; changes from baseline in vaginal pH value at weeks 2, 4, 8, 10, 12, and 16 of treatment; changes from baseline in vaginal maturation index (VMI) at weeks 4, 8, 10, 12 and 16 of treatment; changes from baseline in vaginal sign score (vaginal health index score, VHIS) at weeks 2, 4, 8, 10, 12 and 16 of treatment; and changes from baseline in vaginal colonization of Lactobacillus johnsonii (L. johnsonii), Lactobacillus crispatus (L. crispatus), Lactobacillus jensenii (L. jensenii), and Lactobacillus gasseri (L. gasseri) at weeks 2, 4, 8, 10, 12 and 16 of treatment.

### Safety Endpoints

Adverse events (AEs), serious adverse events (SAEs), clinical laboratory tests, vital signs, physical examinations, gynecological examinations, 12-lead electrocardiograms (ECGs), and other examinations were included.

### Exploratory Endpoints

Changes from baseline in female sexual function index (FSFI) and vaginal aging-related QoL score (DIVA) at week 16 of treatment.

### Example 4: Phase IIb Clinical Trial of Microbial Formulation for Treating Bacterial Vaginosis (BV)

### (I) Design of Trial

This trial employed a multicenter, randomized, double-blind, placebo-controlled design.

Eligible BV patients were randomly grouped according to a ratio of experimental group:control group=1:1 (based on the number of subjects):

Experimental group:

Metronidazole tablets were administered orally twice daily for 7 consecutive days, with 400 mg per administration, and then the live bacteria capsule (prepared in Preparation Example 5 of Example 1) (1.0×10⁹ CFU/capsule) was administered vaginally once daily for 14 consecutive days, with 1 capsule per administration.

Control group:

Metronidazole tablets were administered orally twice daily for 7 consecutive days, with 400 mg per administration, and then the live bacteria capsule mimic (prepared in Example 1) was administered vaginally once daily for 14 consecutive days, with 1 capsule per administration.

The subjects were required to avoid vaginal secretion examinations during the menstrual period. If menstruation occurred after enrollment, the oral administration of the metronidazole tablets could be continued, while the administration of the live bacteria capsule/capsule mimic was paused, and the counting of administration dates was stopped. After the menstruation ended, the subjects continued to take the remaining live bacteria capsule/capsule mimic until the completion of the 14-day administration course. If menstruation occurred at the time point of visit, the corresponding visit was postponed, and the follow-up was completed 3 days after the menstruation ended (the day when the menstruation ended was defined as day 0, with the follow-up performed on day 3).

The subjects were required to attend hospital visits at protocol-specified time points for corresponding procedures and examinations at D−1 to D1, D8±1, D28±4, and D90±7 (for subjects who achieved clinical cure at D28±4 only). If menstruation occurred during any visit period, the visit must be conducted in accordance with the aforementioned visit rules.

(II) Inclusion criteria (subjects who met all of the following conditions were enrolled in this trial): (1) being female of childbearing age with a sexual history, aged 18-55 years (inclusive); (2) being judged by the investigator to have regular menstrual cycles, and being able to use the investigational drug as required by the protocol; (3) at the screening visit, being clinically diagnosed with BV, i.e., having at least 3 items of the Amsel criteria being positive in the clinical diagnosis results (where being positive for clue cells is mandatory), and having a Nugent score≥7 (based on the diagnosis results in our hospital within 3 days prior to signing an informed consent form); (4) having a willingness to have no pregnancy plans and agree to adopt effective contraception measures (no sexual intercourse or consistent condom use for barrier contraception throughout) from screening until 3 months after investigational drug withdrawal; and (5) voluntarily participating in this clinical trial, and being able to comprehend and sign an informed consent form.

(III) Exclusion criteria (subjects who met any one of the following conditions were excluded from this trial): (1) suffering from known current acute urogenital system infections, such as pelvic inflammatory disease, acute cervicitis, urinary tract infections requiring intervention, etc.; (2) suffering from vulvovaginal candidiasis (VVC), vaginal trichomoniasis, suspected gonorrhea, gonorrhea, herpes simplex virus infection, and/or condyloma acuminatum (CA); (3) suffering from other vaginal or vulvar disease conditions that may confound the interpretation of clinical responses; (4) having unexplained vaginal bleeding or known uterine fibroids, endometrial hyperplasia, or adenomyosis considered by the investigator to require intervention; (5) having received local or systemic antimicrobial therapy within 7 days prior to screening, and/or being expected to receive such treatment during the trial; (6) having a history of malignant tumors prior to screening and being considered by the investigator as unsuitable for inclusion; (7) having a history of major gynecological surgery (with deeper wounds requiring prolonged recovery time) within 6 months prior to screening, or having a history of superficial gynecological surgery or common procedures (including but not limited to bartholin cyst incision, bartholin abscess incision, intrauterine device removal (placement), cervical or vaginal wall biopsy, cervical conization, or cervical loop electrosurgical excision procedure) within 60 days prior to screening, or being within 60 days post-termination of pregnancy at the time of screening; (8) having significant vaginal mucosal damage (e.g., mucosal edema, congestion, and ulcer) indicated by gynecological examinations at the time of screening; (9) having alanine aminotransferase (ALT) or aspartate aminotransferase (AST)>2×ULN, serum creatinine (Cr)>1.5×ULN, and blood urea nitrogen (BUN)>1.5×ULN; (10) having any concurrent disease/concomitant surgery/medication/clinically significant abnormal laboratory examination findings that, according to the investigator's judgment, may affect the evaluation of the investigational drug; (11) having known allergy to similar drugs (e.g., Live Lactobacillus Capsule for Vaginal Use) or allergy to metronidazole; (12) being pregnant or breastfeeding women; (13) having participated in any intervention study within 30 days prior to the first administration of this study, or having current or planned enrollment in any other intervention study during participation in this study; and (14) being judged by the investigator to have other medical conditions that may affect the judgment of this trial's results.

### (IV) Baseline Characteristics of Patients

A total of 95 participants were screened in this study, and 80 participants were eligible and successfully enrolled (40 in the experimental group and 40 in the control group). A total of 2 participants withdrew early (1 in each of the experimental group and the control group). All participants completed the treatment phase, and finally 78 participants completed the study. Baseline characteristics were overall balanced between the two groups, demonstrating comparability at baseline.

A total of 80 participants were included in the full analysis set (FAS) in this trial. The following data are based on the FAS.

### (V) Safety and Efficacy Data

### (1) Clinical Cure Rate at D28±4

Note: Clinical cure is defined as having at least 2 out of 4 items of the Amsel criteria being negative in the clinical diagnosis results (where being negative for clue cells is mandatory) and having a Nugent score<7.

At the D8±1 visit, there was no statistical difference in the clinical cure rate between the experimental group and the control group (82.5% VS 92.5%). After supplementary administration of the vaginal live bacteria capsule/capsule mimic, at the D28±4 visit, the clinical cure rate of the experimental group was higher than that of the control group (92.5% VS 60.0%), with a statistical difference (P<0.05). The BV cure status at D28±4 was analyzed using a Logistic regression model. The OR value between the combined experimental group and the control group was 10.097, and the 95% CI was 2.464-41.376 (P=0.001).

TABLE 12
Summary of clinical cure rates of BV at visits after treatment (FAS)

| | | Live bacteria | | |
|---|---|---|---|---|
| | | capsule | Control Group | Total |
| Time | | (N = 40) | (N = 40) | (N = 80) |
| D8 ± 1 | n (nmiss) | 33 (0) | 37 (0) | 70 (0) |
| | BV cure percentage, | 33/40 (82.5) | 37/40 (92.5) | 70/80 (87.5) |
| | n/N1 (%) | | | |
| | 95% confidence | 67.2-92.7 | 79.6-98.4 | 78.2-93.8 |
| | interval | | | |
| | Percentage | −10.0 | | |
| | difference between | | | |
| | experimental group | | | |
| | and control group | | | |
| | 95% confidence | −24.3-4.3 | | |
| | interval | | | |
| | P value | 0.3109 | | |
| D28 ± 4 | n (nmiss) | 37 (1) | 24 (0) | 61 (1) |
| | BV cure percentage, | 37/40 (92.5) | 24/40 (60.0) | 61/80 (76.3) |
| | n/N1 (%) | | | |
| | 95% confidence | 79.6-98.4 | 43.3-75.1 | 65.4-85.1 |
| | interval | | | |
| | Percentage | 32.5 | | |
| | difference between | | | |
| | experimental group | | | |
| | and control group | | | |
| | 95% confidence | 15.3-49.7 | | |
| | interval | | | |
| | P value | 0.0012 | | |

Note:
N is the number of cases in each group's full analysis set, N1 is the number of participants clinically diagnosed with BV at baseline, n is the number of cases with clinical cure of BV, and nmiss is the number of missing cases; clinical cure is defined as having at least 2 items of the Amsel criteria being negative in the clinical diagnosis results (where being negative for clue cells is mandatory) and having a Nugent score <7.
Note:
The 95% confidence intervals for the rates were estimated using the Clopper-Pearson method, and the 95% confidence interval for the difference in rates between the two groups was estimated using the normal approximation method.
Note:
P value calculation is based on Fisher's exact test.

### (2) BV Recurrence Rate at D90±7

Recurrence is defined as having at least 3 out of 4 items of the Amsel criteria being positive (where being positive for clue cells is mandatory) or having a Nugent score≥7.

Cumulative recurrence rate at D90±7: At D90±7, the cumulative recurrence rate was 9.1% ( 3/33) for the experimental group and 45.9% ( 17/37) for the control group. The difference in cumulative recurrence rate between the two groups was −36.9% (95% CI was −55.7% to −18.0%, P=0.0011), indicating that the cumulative recurrence rates of the two groups were statistically different.

TABLE 13
Summary of cumulative BV recurrence rates after treatment (FAS)

| | | Live bacteria | Control | |
|---|---|---|---|---|
| | | capsule | group | Total |
| Time | | (N = 40) | (N = 40) | (N = 80) |
| D90 ± 7 | n (nmiss) | 3 (0) | 17 (0) | 20 (0) |
| | Cumulative | 3/33 (9.1) | 17/37 (45.9) | 20/70 (28.6) |
| | BV recurrence | | | |
| | percentage, n/N1 (%) | | | |
| | 95% confidence | 1.9-24.3 | 29.5-63.1 | 18.4-40.6 |
| | interval | | | |
| | Percentage | −36.9 | | |
| | difference between | | | |
| | experimental | | | |
| | group and control | | | |
| | group | | | |
| | 95% confidence | −55.7-−18.0 | | |
| | interval | | | |
| | P value | 0.0011 | | |

Note:
N is the number of cases in each group's full analysis set, N1 is the number of participants with clinical cure at D8-1, n is the number of cases with cumulative recurrence, and nmiss is the number of missing cases; BV recurrence is defined as having at least 3 out of 4 items of the Amsel criteria being positive in the clinical diagnosis results (where being positive for clue cells is mandatory) and having a Nugent score ≥7.
Note:
The 95% confidence intervals for the rates were estimated using the Clopper-Pearson method, and the 95% confidence interval for the difference in rates between the two groups was estimated using the normal approximation method.
Note:
P value calculation is based on Fisher's exact test.

### (3) Bacterial Cure Rate and Comprehensive Cure Rate

Bacterial cure is defined as having a Nugent score<4;

Comprehensive cure rate is defined as clinical cure and bacterial cure.

At the D8±1 visit, there was no statistical difference in bacterial cure rate (57.5% VS 67.5%) and comprehensive cure rate (57.5% VS 67.5%) between the experimental group and the control group. After supplementary administration of the live bacteria capsule/capsule mimic, at the D28±4 visit, the bacterial cure rate (67.5% VS 50.0%) and the comprehensive cure rate (67.5% VS 50.0%) of the experimental group were higher than those of the control group.

TABLE 14
Summary of bacterial cure rates of BV at visits after treatment (FAS)

| | | Live bacteria | Control | |
|---|---|---|---|---|
| | | capsule | group | Total |
| Time | | (N = 40) | (N = 40) | (N = 80) |
| D8 ± 1 | n (nmiss) | 23 (0) | 27 (0) | 50 (0) |
| | Bacterial cure | 23/40 (57.5) | 27/40 (67.5) | 50/80 (62.5) |
| | percentage, n/N1 | | | |
| | (%) | | | |
| | 95% confidence | 40.9-73.0 | 50.9-81.4 | 51.0-73.1 |
| | interval | | | |
| | Percentage | −10.0 | | |
| | difference between | | | |
| | experimental | | | |
| | group and control | | | |
| | group | | | |
| | 95% confidence | −31.1-11.1 | | |
| | interval | | | |
| | P value | 0.4888 | | |
| D28 ± 4 | n (nmiss) | 27 (1) | 20 (0) | 47 (1) |
| | Bacterial cure | 27/40 (67.5) | 20/40 (50.0) | 47/80 (58.8) |
| | percentage, n/N1 | | | |
| | (%) | | | |
| | 95% confidence | 50.9-81.4 | 33.8-66.2 | 47.2-69.6 |
| | interval | | | |
| | Percentage | 17.5 | | |
| | difference between | | | |
| | experimental | | | |
| | group and control | | | |
| | group | | | |
| | 95% confidence | −3.7-38.7 | | |
| | interval | | | |
| | P value | 0.1726 | | |

Note:
N is the number of cases in each group's full analysis set, N1 is the number of participants clinically diagnosed with BV at baseline, n is the number of cases with clinical cure of BV, and nmiss is the number of missing cases; clinical cure is defined as having at least 2 items of the Amsel criteria being negative in the clinical diagnosis results (where being negative for clue cells is mandatory) and having a Nugent score <7.
Note:
The 95% confidence intervals for the rates were estimated using the Clopper-Pearson method, and the 95% confidence interval for the difference in rates between the two groups was estimated using the normal approximation method.
Note:
P value calculation is based on Fisher's exact test.

TABLE 15
Summary of comprehensive cure rates of BV at visits after treatment
(FAS)

| | | Live bacteria | Control | |
|---|---|---|---|---|
| | | capsule | group | Total |
| Time | | (N = 40) | (N = 40) | (N = 80) |
| D8 ± 1 | n (nmiss) | 23 (0) | 27 (0) | 50 (0) |
| | Comprehensive | 23/40 (57.5) | 27/40 (67.5) | 50/80 (62.5) |
| | cure percentage, | | | |
| | n/N1 (%) | | | |
| | 95% confidence | 40.9-73.0 | 50.9-81.4 | 51.0-73.1 |
| | interval | | | |
| | Percentage | −10.0 | | |
| | difference between | | | |
| | experimental | | | |
| | group and control | | | |
| | group | | | |
| | 95% confidence | −31.1-11.1 | | |
| | interval | | | |
| | P value | 0.4888 | | |
| D28 ± 4 | n (nmiss) | 27 (1) | 20 (0) | 47 (1) |
| | Comprehensive | 27/40 (67.5) | 20/40 (50.0) | 47/80 (58.8) |
| | cure percentage, | | | |
| | n/N1 (%) | | | |
| | 95% confidence | 50.9-81.4 | 33.8-66.2 | 47.2-69.6 |
| | interval | | | |
| | Percentage | 17.5 | | |
| | difference between | | | |
| | experimental | | | |
| | group and control | | | |
| | group | | | |
| | 95% confidence | −3.7-38.7 | | |
| | interval | | | |
| | P value | 0.1726 | | |

Note:
N is the number of cases in each group's full analysis set, N1 is the number of participants clinically diagnosed with BV at baseline, n is the number of cases with comprehensive cure, and nmiss is the number of missing cases; comprehensive cure is defined as having at least 2 out of 4 items of the Amsel criteria being negative in the clinical diagnosis results (where being negative for clue cells is mandatory) and having a Nugent score <4.
Note:
The 95% confidence intervals for the rates were estimated using the Clopper-Pearson method, and the 95% confidence interval for the difference in rates between the two groups was estimated using the normal approximation method.
Note:
P value calculation is based on Fisher's exact test.

### (4) Amsel Criteria, Nugent Score, and Lactobacillus Grading Examination

Amsel criteria, Nugent score, and Lactobacillus grading examination: At the D8±1 visit, there was no statistical difference in Amsel criterion treatment response rate between the experimental group and the control group (85.0% VS 92.5%); at the D28±4 visit, the Amsel criterion treatment response rate was 92.5% ( 37/40) for the experimental group and 60.0% ( 24/40) for the control group; the difference in Amsel criterion treatment response rate between the two groups was 32.5% (95% CI was 15.3% to 49.7%, P=0.0012), indicating that the Amsel response rates of the two groups at D28±4 were statistically different. At the D8±1 visit, there was no statistical difference in Nugent score complete response rate between the experimental group and the control group (57.5% VS 67.5%); at the D28±4 visit, the Nugent score complete response rate was 67.5% ( 27/40) in the experimental group and 50.0% ( 20/40) in the control group; the difference in Nugent score complete response rate between the two groups was 17.5% (95% CI was −3.7 to 38.7, P=0.1726). At the D8±1 visit, there was no statistical difference in Nugent score response rate between the experimental group and the control group (85.0% VS 92.5%); at the D28±4 visit, the Nugent score response rate was 95.0% ( 38/40) for the experimental group and 60.0% ( 24/40) for the control group; the difference in Nugent score response rate between the two groups was 35.0% (95% CI was 18.4 to 51.6, P=0.0003), indicating that the Nugent score response rates of the two groups at D28±4 were statistically different.At the D8±1 visit, there was no statistical difference in Lactobacillus grading response rate between the experimental group and the control group (46.2% VS 55.0%); at the D28±4 visit, the Lactobacillus grading response rate was 61.5% ( 24/39) for the experimental group and 40.0% ( 16/40) for the control group; the difference in Lactobacillus grading response rate between the two groups was 21.5% (95% CI was 0.0 to 43.1), indicating that the Lactobacillus grading response rates of the two groups at D28±4 were not statistically significant.

TABLE 16
Summary of Amsel criterion examination results after treatment-
Amsel criterion treatment response

| | | Live bacteria | Control | |
|---|---|---|---|---|
| | | capsule | Group | Total |
| Time | | (N = 40) | (N = 40) | (N = 80) |
| D8 ± 1 | n (nmiss) | 34 (0) | 37 (0) | 71 (0) |
| | Amsel criterion | 34/40 (85.0) | 37/40 (92.5) | 71/80 (88.8) |
| | treatment | | | |
| | response percentage, | | | |
| | n/N1 (%) | | | |
| | 95% confidence | 70.2-94.3 | 79.6-98.4 | 79.7-94.7 |
| | interval | | | |
| | Percentage | −7.5 | | |
| | difference between | | | |
| | experimental group | | | |
| | and control group | | | |
| | 95% confidence | −21.3-6.3 | | |
| | interval | | | |
| | P value | 0.4814 | | |
| D28 ± 4 | n (nmiss) | 37 (1) | 24 (0) | 61 (1) |
| | Amsel criterion | 37/40 (92.5) | 24/40 (60.0) | 61/80 (76.3) |
| | treatment | | | |
| | response percentage, | | | |
| | n/N1 (%) | | | |
| | 95% confidence | 79.6-98.4 | 43.3-75.1 | 65.4-85.1 |
| | interval | | | |
| | Percentage | 32.5 | | |
| | difference between | | | |
| | experimental group | | | |
| | and control group | | | |
| | 95% confidence | 15.3-49.7 | | |
| | interval | | | |
| | P value | 0.0012 | | |

Note:
N is the number of cases in each group's full analysis set, N1 is the number of participants diagnosed with BV according to the Amsel criteria at baseline, n is the number of cases with treatment response according to the Amsel criteria, and nmiss is the number of missing cases; treatment response is defined as having at least 2 out of 4 items of the Amsel criteria being negative in the clinical diagnosis results (where being negative for clue cells is mandatory).
Note:
The 95% confidence intervals for the rates were estimated using the Clopper-Pearson method, and the 95% confidence interval for the difference in rates between the two groups was estimated using the normal approximation method.
Note:
P value calculation is based on Fisher's exact test.

TABLE 17
Summary of vaginal microecology examination results after
treatment-Nugent score treatment response

| | | | Live | | |
|---|---|---|---|---|---|
| Nugent | | | bacteria | Control | |
| scoring | | | capsule | Group | Total |
| criteria | Time | | (N = 40) | (N = 40) | (N = 80) |
| Nugent | D8 ± 1 | n (nmiss) | 23 (0) | 27 (0) | 50 (0) |
| score | | Nugent score | 23/40 | 27/40 | 50/80 |
| complete | | complete | (57.5) | (67.5) | (62.5) |
| response | | response | | | |
| (<4 | | percentage, | | | |
| points) | | n/N1 (%) | | | |
| | | 95% confidence | 40.9-73.0 | 50.9-81.4 | 51.0-73.1 |
| | | interval | | | |
| | | Percentage | −10.0 | | |
| | | difference | | | |
| | | between | | | |
| | | experimental | | | |
| | | group and | | | |
| | | control | | | |
| | | group | | | |
| | | 95% confidence | −31.1-11.1 | | |
| | | interval | | | |
| | | P value | 0.4888 | | |
| | D28 ± 4 | n (nmiss) | 27 (1) | 20 (0) | 47 (1) |
| | | Nugent score | 27/40 | 20/40 | 47/80 |
| | | complete | (67.5) | (50.0) | (58.8) |
| | | response | | | |
| | | percentage, | | | |
| | | n/N1 (%) | | | |
| | | 95% confidence | 50.9-81.4 | 33.8-66.2 | 47.2-69.6 |
| | | interval | | | |
| | | Percentage | 17.5 | | |
| | | difference | | | |
| | | between | | | |
| | | experimental | | | |
| | | group and | | | |
| | | control | | | |
| | | group | | | |
| | | 95% confidence | −3.7-38.7 | | |
| | | interval | | | |
| | | P value | 0.1726 | | |
| Nugent | D8 ± 1 | n (nmiss) | 34 (0) | 37 (0) | 71 (0) |
| score | | Nugent score | 34/40 | 37/40 | 71/80 |
| response | | response | (85.0) | (92.5) | (88.8) |
| (<7 | | percentage, | | | |
| points) | | n/N1 (%) | | | |
| | | 95% confidence | 70.2-94.3 | 79.6-98.4 | 79.7-94.7 |
| | | interval | | | |
| | | Percentage | 7.5 | | |
| | | difference | | | |
| | | between | | | |
| | | experimental | | | |
| | | group and | | | |
| | | control | | | |
| | | group | | | |
| | | 95% confidence | −21.3-6.3 | | |
| | | interval | | | |
| | | P value | 0.4814 | | |
| | D28 ± 4 | n (nmiss) | 38 (1) | 24 (0) | 62 (1) |
| | | Nugent score | 38/40 | 24/40 | 62/80 |
| | | response | (95.0) | (60.0) | (77.5) |
| | | percentage, | | | |
| | | n/N1 (%) | | | |
| | | 95% confidence | 83.1-99.4 | 43.3-75.1 | 66.8-86.1 |
| | | interval | | | |
| | | Percentage | 35.0 | | |
| | | difference | | | |
| | | between | | | |
| | | experimental | | | |
| | | group and | | | |
| | | control | | | |
| | | group | | | |
| | | 95% confidence | 18.4-51.6 | | |
| | | interval | | | |
| | | P value | 0.0003 | | |
| Lacto- | D8 ± 1 | n (nmiss) | 18 (0) | 22 (0) | 40 (0) |
| bacillus | | Lactobacillus | 18/39 | 22/40 | 40/79 |
| response | | treatment | (46.2) | (55.0) | (50.6) |
| | | response | | | |
| | | percentage, | | | |
| | | n/N1 (%) | | | |
| | | 95% confidence | 30.1-62.8 | 38.5-70.7 | 39.1-62.1 |
| | | interval | | | |
| | | Percentage | −8.8 | | |
| | | difference | | | |
| | | between | | | |
| | | experimental | | | |
| | | group and | | | |
| | | control | | | |
| | | group | | | |
| | | 95% confidence | −30.8-13.1 | | |
| | | interval | | | |
| | | P value | 0.5026 | | |
| | D28 ± 4 | n (nmiss) | 24 (1) | 16 (0) | 40 (1) |
| | | Lactobacillus | 24/39 | 16/40 | 40/79 |
| | | treatment | (61.5) | (40.0) | (50.6) |
| | | response | | | |
| | | percentage, | | | |
| | | n/N1 (%) | | | |
| | | 95% confidence | 44.6-76.6 | 24.9-56.7 | 39.1-62.1 |
| | | interval | | | |
| | | Percentage | 21.5 | | |
| | | difference | | | |
| | | between | | | |
| | | experimental | | | |
| | | group and | | | |
| | | control | | | |
| | | group | | | |
| | | 95% confidence | 0.0-43.1 | | |
| | | interval | | | |
| | | P value | 0.0729 | | |

Note:
N is the number of cases in each group's full analysis set, N1 is the number of participants with scores greater than or equal to the corresponding score at baseline, n is the number of cases with scores less than the corresponding score, and nmiss is the number of missing cases.
Note:
The 95% confidence intervals for the rates were estimated using the Clopper-Pearson method, and the 95% confidence interval for the difference in rates between the two groups was estimated using the normal approximation method.
Note:
P value calculation is based on Fisher's exact test.

### (5) 16S RDNA Gene Sequencing Was Used to Evaluate the Changes in the Relative Abundance of Lactobacillus

Changes in relative abundance of Lactobacillus genus

16S rDNA gene sequencing was used to evaluate changes in the relative abundance and quantity of vaginal microbiota in vaginal secretions.

Before and after medication administration, the average relative abundance of Lactobacillus genus in the experimental group and control group at each visit point is shown in Table 18.

TABLE 18
The relative abundance of vaginal Lactobacillus at each visit point
(Mean ± SD)

| Group | D-1~D1 | D8 ± 1 | D28 ± 4 | D90 ± 7 |
|---|---|---|---|---|
| Experimental | 9% ± 18% | 61% ± 37% | 76% ± 34% | 74% ± 36% |
| group | | | | |
| Control group | 12% ± 22% | 69% ± 31% | 57% ± 43% | 76% ± 34% |
| P | 0.9446 | 0.4909 | 0.0206 | 0.7528 |

After excluding Lactobacillus iners, further analysis is shown in Table 19. It revealed that supplementation with microbial formulation of the present disclosure can increase the relative abundance of vaginal Lactobacillus other than Lactobacillus iners in BV patients.

TABLE 19
The relative abundance of vaginal Lactobacillus (other than
Lactobacillus iners) at each visit point (Mean ± SD)

| Group | D-1~D1 | D8 ± 1 | D28 ± 4 | D90 ± 7 |
|---|---|---|---|---|
| Experimental | 1% ± 4% | 17% ± 31% | 48% ± 39% | 42% ± 43% |
| group | | | | |
| Control group | 2% ± 12% | 7% ± 21% | 5% ± 16% | 17% ± 35% |
| P | 0.9639 | 0.2082 | 0.0000 | 0.0104 |

Changes in relative abundance of individual Lactobacillus (Lactobacillus crispatus, Lactobacillus jensenii, Lactobacillus gasseri, Lactobacillus johnsonii and Lactobacillus inerts)

Before and after medication administration, the average relative abundance of individual Lactobacillus (Lactobacillus crispatus, Lactobacillus jensenii, Lactobacillus gasseri, Lactobacillus johnsonii, and Lactobacillus inerts) in the experimental group and control group at each visit point is shown in Table 20.

TABLE 20
The relative abundance of individual Lactobacillus (Lactobacillus crispatus, Lactobacillus jensenii,
Lactobacillus gasseri, Lactobacillus
johnsonii, and Lactobacillus inerts) at each visit point (Mean ± SD)

| Species | Groups | D-1~D1 | D8 ± 1 | D28 ± 4 | D90 ± 7 |
|---|---|---|---|---|---|
| Lactobacillus | Experimental | 1% ± 3% | 7% ± 18% | 28% ± 35% | 36% ± 42% |
| crispatus | group | | | | |
| | Control | 2% ± 11% | 5% ± 20% | 4% ± 15% | 16% ± 34% |
| | group | | | | |
| | P | 0.6986 | 0.6833 | 0.0000 | 0.1786 |
| Lactobacillus | Experimental | 0% ± 2% | 4% ± 10% | 15% ± 25% | 6% ± 17% |
| jensenii | group | | | | |
| | Control | 0% ± 1% | 2% ± 8% | 2% ± 4% | 1% ± 2% |
| | group | | | | |
| | P | 0.0496 | 0.2655 | 0.0000 | 0.0114 |
| Lactobacillus | Experimental | 0% ± 0% | 5% ± 17% | 5% ± 16% | 0% ± 1% |
| gasseri | group | | | | |
| | Control | 0% ± 0% | 0% ± 0% | 0% ± 0% | 0% ± 1% |
| | group | | | | |
| | P | 0.9655 | 0.0160 | 0.0000 | 0.5315 |
| Lactobacillus | Experimental | 0% ± 0% | 1% ± 2% | 0% ± 1% | 0% ± 0% |
| johnsonii | group | | | | |
| | Control | 0% ± 0% | 0% ± 0% | 0% ± 0% | 0% ± 0% |
| | group | | | | |
| | P | 0.3379 | 0.3496 | 0.3759 | 0.5579 |
| Lactobacillus | Experimental | 8% ± 16% | 44% ± 39% | 28% ± 31% | 32% ± 37% |
| inerts | group | | | | |
| | Control | 10% ± 19% | 62% ± 33% | 52% ± 41% | 59% ± 41% |
| | group | | | | |
| | P | 0.7004 | 0.0859 | 0.0033 | 0.0056 |

In summary, the above results indicate that supplementation with microbial formulation of the present disclosure can increase the relative abundance of vaginal Lactobacillus other than Lactobacillus iners and decrease the relative abundance of Lactobacillus iners in BV patients, thereby transforming the vaginal microbiota into a healthier one.

### (6) Safety Data

The adverse events and adverse reactions in this trial are summarized in Tables 21 and 22. In this clinical trial, the overall incidence of adverse events was 66.3% ( 53/80, 102 cases), and all the adverse events were grade 1 (41.3%, 33/80) or grade 2 (41.3%, 33/80); the incidence of adverse events was 60.0% ( 24/80, 57 cases) for the experimental group and 72.5% ( 29/40, 45 cases) for the control group. No adverse events leading to withdrawal from the trial occurred, and no adverse events with CTCAE≥grade 3 occurred. There were no SAEs leading to death; and no serious adverse events (SAEs) occurred. Based on SS, the incidence of adverse reactions was 35.0% ( 28/80, 35 cases), and all the adverse reactions were grade 1 (21.3%, 17/80) or grade 2 (17.5%, 14/80) and finally showed an outcome of recovery. The incidence of adverse reactions was 40.0% ( 16/40, 23 cases) for the experimental group and 30.0% ( 12/40, 12 cases) for the control group. The adverse reactions related to the live bacteria capsule had an incidence of 15.0% ( 6/40, 8 cases) and, when classified by PT, included: vulvovaginal candidiasis (10%, 4/40, 4 cases), vulvovaginal pruritus (7.5%, 3/40, 3 cases), and abnormal uterine bleeding (2.5%, 1/40, 1 case). No adverse reactions leading to withdrawal from the trial occurred, and no adverse reactions with CTCAE≥grade 3 occurred. During the treatment period, there were no serious adverse reactions (SARs); and no suspected unexpected serious adverse reactions (SUSARs) occurred.

TABLE 21
Summary of adverse events (SS)

| | Live bacteria | Control | | |
|---|---|---|---|---|
| | capsule | group | Total | |
| | (N = 40) | (N = 40) | (N = 80) | P |
| | n (%) E | n (%) E | n (%) E | value |
| All adverse events | 24 (60.0) 57 | 29 (72.5) 45 | 53 (66.3) 102 | 0.3444 |
| TEAE | 24 (60.0) 57 | 29 (72.5) 45 | 53 (66.3) 102 | 0.3444 |
| TEAE leading to | 0 | 0 | 0 | NE |
| withdrawal from trial | | | | |
| TEAE leading to | 1 (2.5) 1 | 0 | 1 (1.3) 1 | 1.0000 |
| measures taken on | | | | |
| investigational drug | | | | |
| TEAE leading to | 1 (2.5) 1 | 0 | 1 (1.3) 1 | 1.0000 |
| measures taken on | | | | |
| live bacteria | | | | |
| capsule/capsule mimic | | | | |
| TEAE leading to | 0 | 0 | 0 | NE |
| measures taken on | | | | |
| metronidazole tablets | | | | |
| TEAE CTCAE grade | | | | |
| Grade 1 | 17 (42.5) 33 | 16 (40.0) 24 | 33 (41.3) 57 | 1.0000 |
| Grade 2 | 16 (40.0) 24 | 17 (42.5) 21 | 33 (41.3) 45 | 1.0000 |
| Grade 3 | 0 | 0 | 0 | NE |
| Grade 4 | 0 | 0 | 0 | NE |
| Grade 5 | 0 | 0 | 0 | NE |
| TEAE with | 0 | 0 | 0 | NE |
| CTCAE ≥ grade 3 | | | | |
| Maximum severity | | | | |
| grade of TEAE | | | | |
| Grade 1 | 8 (20.0) | 12 (30.0) | 20 (25.0) | 0.4391 |
| Grade 2 | 16 (40.0) | 17 (42.5) | 33 (41.3) | 1.0000 |
| Grade 3 | 0 | 0 | 0 | NE |
| Grade 4 | 0 | 0 | 0 | NE |
| Grade 5 | 0 | 0 | 0 | NE |
| TEAE with | 0 | 0 | 0 | NE |
| CTCAE ≥ grade 3 | | | | |
| TRAE | 16 (40.0) 23 | 12 (30.0) 12 | 28 (35.0) 35 | 0.4823 |
| TRAE leading to | 0 | 0 | 0 | NE |
| withdrawal from trial | | | | |
| TRAE leading to | 0 | 0 | 0 | NE |
| measures taken on | | | | |
| investigational drug | | | | |
| TRAE leading to | 0 | 0 | 0 | NE |
| measures taken on | | | | |
| live bacteria | | | | |
| capsule/capsule mimic | | | | |
| TRAE leading to | 0 | 0 | 0 | NE |
| measures taken on | | | | |
| metronidazole tablets | | | | |
| TRAE CTCAE grade | | | | |
| Grade 1 | 11 (27.5) 14 | 6 (15.0) 6 | 17 (21.3) 20 | 0.2742 |
| Grade 2 | 8 (20.0) 9 | 6 (15.0) 6 | 14 (17.5) 15 | 0.7695 |
| Grade 3 | 0 | 0 | 0 | NE |
| Grade 4 | 0 | 0 | 0 | NE |
| Grade 5 | 0 | 0 | 0 | NE |
| TEAE with | 0 | 0 | 0 | NE |
| CTCAE ≥ grade 3 | | | | |
| Maximum severity | | | | |
| grade of TRAE | | | | |
| Grade 1 | 8 (20.0) | 6 (15.0) | 14 (17.5) | 0.7695 |
| Grade 2 | 8 (20.0) | 6 (15.0) | 14 (17.5) | 0.7695 |
| Grade 3 | 0 | 0 | 0 | NE |
| Grade 4 | 0 | 0 | 0 | NE |
| Grade 5 | 0 | 0 | 0 | NE |
| TRAE with | 0 | 0 | 0 | NE |
| CTCAE ≥ grade 3 | | | | |
| TEAE leading to death | 0 | 0 | | |
| SAE | 0 | 0 | | |
| All SAEs related | 0 | 0 | | |
| to investigational | | | | |
| drug or control drug | | | | |
| Suspected unexpected | 0 | 0 | | |
| serious adverse | | | | |
| reaction (SUSAR) | | | | |

Note:
N is the number of cases in each group's safety analysis set; n is the number of participants with at least one adverse event occurrence; % is the percentage of participants with at least one adverse event occurrence; and E is the number of adverse event occurrences.
Note:
The treatment-emergent adverse event (TEAE) is defined as an AE that occurs after the first administration (including the same day).
Note:
The adverse event related to investigational drug or control drug (treatment-related adverse event, TRAE) is defined as an adverse event that, per the adverse event collection page of CRF, is categorized as “related, probably related, or possibly related” to the investigational drug or control drug.
Note:
The measures taken on the investigational drug include: permanent withdrawal, temporary interruption, and unknown.
Note:
When the incidence rates of adverse events are summarized, if the same adverse event occurs multiple times in the same participant, unless otherwise specified, it is counted as one case and its severity is recorded based on the most severe occurrence.
Note:
P value calculation is based on Fisher's exact test.

TABLE 22
Number and frequency of occurrence of adverse reactions related to
live bacteria capsule/capsule mimic after administration (SS)

| | Live bacteria | Control | | |
|---|---|---|---|---|
| | capsule | group | Total | |
| System organ | (N = 40) | (N = 40) | (N = 80) | |
| preferred term | n (%) E | n (%) E | n (%) E | P value |
| Adverse event | 6 (15.0) 8 | 4 (10.0) 4 | 10 (12.5) 12 | 0.7370 |
| related to live bacteria | | | | |
| capsule/capsule | | | | |
| mimic during treatment | | | | |
| Infection and infestation | 4 (10.0) 4 | 3 (7.5) 3 | 7 (8.8) 7 | 1.0000 |
| Vulvovaginal candidiasis | 4 (10.0) 4 | 1 (2.5) 1 | 5 (6.3) 5 | 0.3589 |
| Escherichia vaginitis | 0 | 1 (2.5) 1 | 1 (1.3) 1 | 1.0000 |
| Genital folliculitis | 0 | 1 (2.5) 1 | 1 (1.3) 1 | 1.0000 |
| Reproductive system | 4 (10.0) 4 | 1 (2.5) 1 | 5 (6.3) 5 | 0.3589 |
| and breast diseases | | | | |
| Vulvovaginal pruritus | 3 (7.5) 3 | 0 | 3 (3.8) 3 | 0.2405 |
| Abnormal uterine | 1 (2.5) 1 | 0 | 1 (1.3) 1 | 1.0000 |
| bleeding | | | | |
| Vaginal secretion | 0 | 1 (2.5) 1 | 1 (1.3) 1 | 1.0000 |

Note:
N is the number of cases in each group's safety analysis set; n is the number of participants with at least one adverse event occurrence; % is the percentage of participants with at least one adverse event occurrence; and E is the number of adverse event occurrences.
Note:
The adverse event related to investigational drug or control drug (treatment-related adverse event, TRAE) is defined as an adverse event that, per the adverse event collection page of CRF, is categorized as “related, probably related, or possibly related” to the investigational drug or control drug.
Note:
When the incidence rates of adverse events are summarized, if the same adverse event occurs multiple times in the same participant, unless otherwise specified, it is counted as one case.

From the perspective of safety, all the adverse reactions caused by the live bacteria capsule were grade 1-2 and finally showed an outcome of recovery, and there was no statistical difference in the incidence of adverse reactions between the experimental group and the placebo control group, indicating that the live bacteria capsule exhibited good safety.

### Conclusion

In summary, the live bacteria capsule can significantly improve the cure rate of patients with bacterial vaginosis (BV); when used sequentially with an antibiotic, the live bacteria capsule can improve the clinical cure rate to no less than 90%, and can also reduce the recurrence rate; the 3-month recurrence rate is less than 10%, representing a reduction of no less than 30% compared to antibiotic monotherapy; additionally, the live bacteria capsule exhibits good safety. Therefore, the live bacteria capsule shows surprising clinical application potential.

It can be known from the general technical knowledge that the present disclosure can be implemented by other embodiments without departing from the spirit or essential features thereof. Therefore, the embodiments disclosed above are, in every respect, merely illustrative and not exclusive. All modifications within the scope of the present disclosure or within the scope equivalent to the present disclosure are encompassed in the present disclosure.

1. A method for improving vaginal health status, comprising administering to a subject a therapeutically effective amount of a microbial formulation, wherein the microbial formulation comprises a probiotic composition comprising active ingredients comprising Lactobacillus johnsonii, Lactobacillus gasseri, Lactobacillus crispatus, and Lactobacillus jensenii, and the improving vaginal health status is selected from:
a): improving the therapeutic effect of an additional drug for treating bacterial vaginosis in the treatment of bacterial vaginosis;
b): improving the prognosis of bacterial vaginosis treated with an additional drug for treating bacterial vaginosis;
c): treating, preventing, mitigating, and/or controlling the recurrence of bacterial vaginosis in the subject;
d): treating, preventing, mitigating, and/or controlling postmenopausal atrophic vaginitis in the subject;
e): improving the Nugent score of the subject;
f): ameliorating vaginal dryness in the subject; and
g): regulating the vaginal microbiota of the subject.

2. The method according to claim 1, wherein the microbial formulation is used in combination with an additional drug for treating bacterial vaginosis, the additional drug for treating bacterial vaginosis being selected from an antibiotic, boric acid, and/or a traditional Chinese medicine formulation.

3. The method according to claim 2, wherein the microbial formulation is used in combination with an antibiotic for treating bacterial vaginosis.

4. The method according to claim 3, wherein the antibiotic is selected from one or more of metronidazole, tinidazole, ornidazole, clindamycin, and fluconazole.

5. The method according to claim 2, wherein the additional drug for treating bacterial vaginosis is administered to the subject prior to the administration of the microbial formulation to the subject.

6. The method according to claim 5, wherein an antibiotic treatment is first administered to the subject, and after the antibiotic treatment is completed, the microbial formulation is administered to the subject for 10-18 consecutive days, with each of the strains in the microbial formulation administered in an amount of 10⁶ CFU to 10¹³ CFU per day.

7. The method according to claim 5, wherein an antibiotic treatment is first administered to the subject, and after the antibiotic treatment is completed, the microbial formulation is administered to the subject once daily for 10-18 consecutive days, preferably 14 consecutive days, with each of the strains administered in an amount of 10⁶ CFU to 10¹³ CFU per administration.

8. The method according to claim 1, wherein the Lactobacillus johnsonii is: Lactobacillus johnsonii with a microbial deposit number of CCTCC M 2019426, a pure culture thereof, a passaged strain thereof, or a clonal strain thereof; or Lactobacillus johnsonii having 96% or more, 97% or more, 98% or more, or 99% or more ANI and substantially identical genetic characteristics to the strain with the microbial deposit number of CCTCC M 2019426; or Lactobacillus johnsonii having a substantially identical genomic sequence to the strain with the microbial deposit number of CCTCC M 2019426;
the Lactobacillus gasseri is: Lactobacillus gasseri with a microbial deposit number of CCTCC M 2019430, a pure culture thereof, a passaged strain thereof, or a clonal strain thereof; or Lactobacillus gasseri having 96% or more, 97% or more, 98% or more, or 99% or more ANI and substantially identical genetic characteristics to the strain with the microbial deposit number of CCTCC M 2019430; or Lactobacillus gasseri having a substantially identical genomic sequence to the strain with the microbial deposit number of CCTCC M 2019430;
the Lactobacillus crispatus is: Lactobacillus crispatus with a microbial deposit number of CCTCC M 2019427, a pure culture thereof, a passaged strain thereof, or a clonal strain thereof; or Lactobacillus crispatus having 96% or more, 97% or more, 98% or more, or 99% or more ANI and substantially identical genetic characteristics to the strain with the microbial deposit number of CCTCC M 2019427; or Lactobacillus crispatus having a substantially identical genomic sequence to the strain with the microbial deposit number of CCTCC M 2019427;
the Lactobacillus jensenii is: Lactobacillus jensenii with a microbial deposit number of CCTCC M 2019429, a pure culture thereof, a passaged strain thereof, or a clonal strain thereof; or Lactobacillus jensenii having 96% or more, 97% or more, 98% or more, or 99% or more ANI and substantially identical genetic characteristics to the strain with the microbial deposit number of CCTCC M 2019429; or Lactobacillus jensenii having a substantially identical genomic sequence to the strain with the microbial deposit number of CCTCC M 2019429.

9. The method according to claim 1, wherein the subject is a human female.

10. The method according to claim 1, wherein based on CFU, in the probiotic composition, any two of the bacteria are in an amount ratio of (1-100):(1-100), preferably (1-10):(1-10), and more preferably 1:(1-10).

11. The method according to claim 1, wherein in a unit formulation of the microbial formulation, the total viable count of the bacteria is not less than 1×10⁷ CFU, and the viable count of each of the strains is not less than 1×10⁶ CFU.

12. The method according to claim 1, wherein the microbial formulation further comprises a pharmaceutically acceptable excipient.

13. The method according to claim 1, wherein the probiotic composition is a lyophilized powder.

14. The method according to claim 1, wherein the microbial formulation is in a dosage form selected from: a capsule, a tablet, and a lyophilized powder.

15. The method according to claim 1, wherein in a unit formulation of the microbial formulation, the viable count of each of the strains is 1×10⁶ CFU to 1×10¹³ CFU.

16. The method according to claim 1, wherein in a unit formulation of the microbial formulation, the viable count of each of the strains is (1-10)×10⁶ CFU, or (1-10)×10⁷ CFU, or (1-10)×10⁸ CFU, or (1-10)×10⁹ CFU, or (1-10)×10¹⁰ CFU, or (1-10)×10¹¹ CFU, or (1-10)×10¹² CFU.

17. The method according to claim 1, wherein regulating the vaginal microbiota of the subject comprises increasing the relative abundance of vaginal Lactobacillus, increasing the relative abundance of vaginal Lactobacillus other than Lactobacillus iners, and/or decreasing the relative abundance of vaginal Lactobacillus iners in the subject.

18. A probiotic composition, comprising active ingredients including Lactobacillus johnsonii, Lactobacillus gasseri, Lactobacillus crispatus, and Lactobacillus jensenii, wherein the probiotic composition is a lyophilized powder.

19. The probiotic composition according to claim 18, wherein the Lactobacillus johnsonii is: Lactobacillus johnsonii with a microbial deposit number of CCTCC M 2019426, a pure culture thereof, a passaged strain thereof, or a clonal strain thereof; or Lactobacillus johnsonii having 96% or more, 97% or more, 98% or more, or 99% or more ANI and substantially identical genetic characteristics to the strain with the microbial deposit number of CCTCC M 2019426; or Lactobacillus johnsonii having a substantially identical genomic sequence to the strain with the microbial deposit number of CCTCC M 2019426;
the Lactobacillus gasseri is: Lactobacillus gasseri with a microbial deposit number of CCTCC M 2019430, a pure culture thereof, a passaged strain thereof, or a clonal strain thereof; or Lactobacillus gasseri having 96% or more, 97% or more, 98% or more, or 99% or more ANI and substantially identical genetic characteristics to the strain with the microbial deposit number of CCTCC M 2019430; or Lactobacillus gasseri having a substantially identical genomic sequence to the strain with the microbial deposit number of CCTCC M 2019430;
the Lactobacillus crispatus is: Lactobacillus crispatus with a microbial deposit number of CCTCC M 2019427, a pure culture thereof, a passaged strain thereof, or a clonal strain thereof; or Lactobacillus crispatus having 96% or more, 97% or more, 98% or more, or 99% or more ANI and substantially identical genetic characteristics to the strain with the microbial deposit number of CCTCC M 2019427; or Lactobacillus crispatus having a substantially identical genomic sequence to the strain with the microbial deposit number of CCTCC M 2019427;
the Lactobacillus jensenii is: Lactobacillus jensenii with a microbial deposit number of CCTCC M 2019429, a pure culture thereof, a passaged strain thereof, or a clonal strain thereof; or Lactobacillus jensenii having 96% or more, 97% or more, 98% or more, or 99% or more ANI and substantially identical genetic characteristics to the strain with the microbial deposit number of CCTCC M 2019429; or Lactobacillus jensenii having a substantially identical genomic sequence to the strain with the microbial deposit number of CCTCC M 2019429.

20. A microbial formulation, comprising the Lactobacillus johnsonii, the Lactobacillus gasseri, the Lactobacillus crispatus, and the Lactobacillus jensenii according to claim 19, with each strain having a viable count of 1×10⁶ CFU to 1×10¹³ CFU per unit formulation.
